(19) United States
(12) Patent Application Publication
(10) Pub. No.: US 2024/0138661 A1
(43) Pub. Date: May 2, 2024

(54) DIVERTER AND/OR AEROSOLIZATION PREVENTER

(71) Applicant: Grumpy Innovation, Inc., New York City, NY (US)

(72) Inventors: Sameer SHARMA, Sioux Falls, SD (US); Luke Emerson HESS, Bemidji, MN (US)

(73) Assignee: Grumpy Innovation, Inc., New York City, NY (US)

(21) Appl. No.: 18/404,703

(22) Filed: Jan. 4, 2024

Related U.S. Application Data
(63) Continuation of application No. PCT/US2022/073609, filed on Jul. 11, 2022.
(60) Provisional application No. 63/220,179, filed on Jul. 9, 2021.

(51) Int. Cl. A61B 1/015; A61B 1/00

(52) U.S. Cl. CPC A61B 1/015; A61B 1/00068; A61B 1/0014

(57) ABSTRACT

A flow diverter is disclosed. The flow diverter can be removably securable to a medical device. The flow diverter can have a body and a cavity. When the body is removably secured to the medical device and when a flow of gas flows from the medical device into the cavity, the flow of gas can be divertible from a first direction to a second direction, and/or when the body is removably secured to the medical device and when the flow of gas flows from the medical device into the cavity, the flow of gas can be divertible out of the cavity.

### CROSS-REFERENCE TO RELATED APPLICATIONS

This application is a continuation of International Patent Application No. PCT/US2022/073609 filed Jul. 11, 2022, which claims the benefit of priority to U.S. Provisional Application No. 63/220,179 filed Jul. 9, 2021, each of which is incorporated herein by reference in its entirety for all purposes.

### BACKGROUND

### 1. Technical Field

This disclosure relates generally to diverters, aerosolization preventers, and/or rests that can be removably attached to medical devices, and more particularly to the use of diverters, aerosolization preventers and/or rests with endoscopes.

### 2. Background

Under experimental circumstances, SARS-CoV-2 has exhibited aerosol transmission and remains viable and infectious up to 3 hours. Currently, a growing body of published evidence points towards endoscopic procedures being considered as aerosol-generating.

In addition to esophagogastroduodenoscopy (EGD), endoscopic retrograde cholangiopancreatography (ERCP) and colonoscopic procedures have a risk of infection, considering the recent detection of the SARS-CoV-2 in specimens and feces. Contaminated droplets and aerosols originate mainly from the patients' upper or lower gastrointestinal tract and previous reports have mainly focused on preventing the spread of these aerosols.

Contaminated aerosolization emanating directly from a flexible endoscope device (e.g., gas/water, suction and biopsy valves) or endoscopic tools has been explored. Endoscopic tools used during flexible endoscopy permit gas leakage from the scope and tools. Indeed the endoscope handle and tools have been shown to cause aerosolization of endoscopic gas containing pathogenic contaminants (as these areas are close to the operators' and assistants' faces during a procedure).

A need exists for a device that would significantly diminish or prevent aerosolization of endoscopic gas. Current methods require PPE or similar techniques. It would be advantageous to be able to place a device over and around the leakage areas of the devices during the procedure to prevent gas leakage.

A need exists for a device (e.g., the device 100 disclosed herein) that allows the selective covering of aerosolization liable areas but still allows full functionality of the endoscope functions whilst protecting the endoscope user and others.

A need exists for a flow diverter.

A need exists for an aerosolization preventor.

A need exists for an aerosolization preventor with an adjustable sealing strap that can inhibit or prevent gas aerosolization.

A need exists for positional placement of an aerosolization preventor using a selectively applied sealing strap to allow further prevention of gas aerosolization.

A need exists for fully sterile single use materials.

A need exists for improved devices (e.g., the device 100 disclosed herein) to improve endoscopic handling ergonomics. Ergonomics in endoscopy has begun to be of interest in the last decade. This has been in response to an increased number of injuries that have been reported due to endoscopy. Studies have cited as high as 89% of endoscopists reporting some type of endoscopy-related injury or pain with hand/finger injuries being the most prevalent in almost all studies. Many have gone through great efforts to mitigate injury through ergonomic conducive endoscopic suite preparation, but little has been done to improve ergonomics of the endoscope itself.

A need exists for a device (e.g., the device 100 disclosed herein) with improved ergonomic features that can recruit muscles of the arm and shoulder to minimize the load sustained by the hand/wrist. The device can be fixed to a scope (e.g., an endoscope) with straps. The material used can be firm enough to sustain the load of the scope but can have a soft outer finish to maximize comfort for the user

### SUMMARY

Covers are disclosed. Flow diverters are disclosed. Covers that can divert flow are disclosed. Aerosolization preventors are disclosed. Covers that can inhibit or prevent aerosolization are disclosed. Flow diverters that can prevent aerosolization are disclosed. Handles are disclosed. Ergonomic handles are disclosed. Handles that can inhibit or prevent the formation of overuse injuries from the use of endoscopes are disclosed.

A flow diverter is disclosed. The flow diverter can be removably securable to a medical device. The flow diverter can have a body and a cavity. When the body is removably secured to the medical device and when a flow of gas flows from the medical device into the cavity, the flow of gas can be divertible from a first direction to a second direction, and/or when the body is removably secured to the medical device and when the flow of gas flows from the medical device into the cavity, the flow of gas can be divertible out of the cavity.

A flow diverter is disclosed. The flow diverter can be removably securable to a medical device. The flow diverter can have a body and a cavity. The body and the cavity have a non-deflected configuration and a deflected configuration. When the body is removably secured to the medical device and when the body and the cavity are in the non-deflected configuration, a first flow path can extend from the cavity, a second flow path can extend from the cavity, and/or a third flow path can extend from the cavity.

A flow diverter is disclosed. The flow diverter can be removably securable to a medical device. The flow diverter can have a body and a cavity. When the body is removably secured to the medical device and when a flow of gas flows from the medical device into the cavity, the flow of gas can be selectively divertible into a first flow path, into a second flow path, and/or into a third flow path.

A flow diverter is disclosed. The flow diverter can be removably securable to a medical device. The flow diverter can have a body and a cavity. When the body is removably secured to the medical device, a first flow path is openable and closable, a second flow path is openable and closable, and/or a third flow path is openable and closable.

A flow diverter is disclosed. The flow diverter can be removably securable to a medical device. The flow diverter can have a body and a cavity. When the body is removably secured to the medical device, the body can be configured to divert a flow of gas from the medical device from a first direction into a second direction, and/or when the body is removably secured to the medical device, the body can be configured to divert the flow of gas the flow of gas out of the cavity.

A flow diverter is disclosed. The flow diverter can be removably securable to a medical device. The flow diverter can have a body and a cavity. When the body is removably secured to the medical device, the body can be configured to divert a flow of gas from the medical device into a first flow, into a second flow, and/or into a third flow.

An aerosolization prevention system is disclosed that can have a flow diverter and a strap. The flow diverter can be removably attached to a medical device via a strap.

A method of diverting a flow of gas from a medical device is disclosed. The method can include attaching a flow diverter to the medical device.

A method of inhibiting or preventing aerosolization is disclosed. The method can include attaching a flow diverter to a medical device.

An ergonomic rest is disclosed. The ergonomic rest can be attachable to an endoscope. The ergonomic rest can be configured to prevent overuse injuries.

### BRIEF SUMMARY OF THE DRAWINGS

The drawings shown and described are exemplary variations and non-limiting Like reference numerals indicate identical or functionally equivalent features throughout.

FIG. 1 illustrates a top view of a variation of a diverter.

FIG. 2 illustrates a bottom perspective view of the diverter of FIG. 1.

FIG. 3 illustrates a top perspective view of the diverter of FIG. 1.

FIG. 4 illustrates a top perspective view of a variation of a strap (e.g., for use with the diverter of FIG. 1).

FIG. 5 illustrates a top perspective view of a variation of a strap (e.g., for use with the diverter of FIG. 1).

FIG. 6 illustrates the diverter of FIG. 1 attached to a variation of an endoscope using two straps of FIG. 4.

FIG. 7 illustrates the endoscope of FIG. 6 without the diverter attached to the endoscope.

FIG. 8 illustrates a variation of the endoscope of FIG. 6.

FIG. 9 illustrates a variation of a close-up view of a distal end of the endoscope of FIG. 8 at section 9x.

FIG. 10 illustrates a variation of a schematic of an endoscopy system.

FIG. 11 illustrates a variation of a schematic of an endoscopy system.

FIG. 12 illustrates a variation of the endoscope of FIG. 6 when an opening on a gas/water button is not covered and when a suction button is not pressed.

FIG. 13A illustrates a side view of the suction button of the endoscope of FIG. 6 in a non-pressed state.

FIG. 13B illustrates a side view of the suction button of the endoscope of FIG. 6 in a pressed state.

FIG. 14A illustrates a side view of a diverter attached to an endoscope when the diverter is in a non-deflected configuration.

FIG. 14B illustrates the side view of FIG. 14A when the diverter is in a deflected configuration.

FIG. 15A illustrates a side view of a diverter attached to an endoscope when the diverter is in a non-deflected configuration.

FIG. 15B illustrates the side view of FIG. 15A when the diverter is in a deflected configuration.

FIG. 16A illustrates a side view of a diverter attached to an endoscope when the diverter is in a non-deflected configuration.

FIG. 16B illustrates the side view of FIG. 16A when the diverter is in a deflected configuration.

FIG. 17A illustrates a side view of the diverter of FIG. 1 attached to an endoscope when the diverter is in a non-deflected configuration.

FIG. 17B illustrates a top view of the diverter and the endoscope of FIG. 17A.

FIG. 18 illustrates a top perspective view of the diverter of FIG. 1 attached to an endoscope.

FIG. 19A illustrates a side view of the diverter of FIG. 1 attached to an endoscope when the diverter is in a non-deflected configuration.

FIG. 19B illustrates a top view of the diverter and the endoscope of FIG. 19A.

FIG. 20A illustrates a side view of the diverter of FIG. 19A attached to an endoscope when the diverter is in a non-deflected configuration.

FIG. 20B illustrates the side view of FIG. 20A when the diverter is in a deflected configuration.

FIG. 21 illustrates a side view of a variation of a diverter attached to an endoscope.

FIG. 22A illustrates a side view of the diverter of FIG. 19A attached to an endoscope when the diverter is in a non-deflected configuration.

FIG. 22B illustrates the side view of FIG. 22A when the diverter is in a deflected configuration.

FIG. 23A illustrates a side view of a variation of an ergonomic rest attached to an endoscope.

FIG. 23B illustrates a perspective view of the ergonomic rest of FIG. 23A.

FIG. 23C illustrates a user operating an endoscope with the ergonomic rest in FIG. 23A.

### DETAILED DESCRIPTION

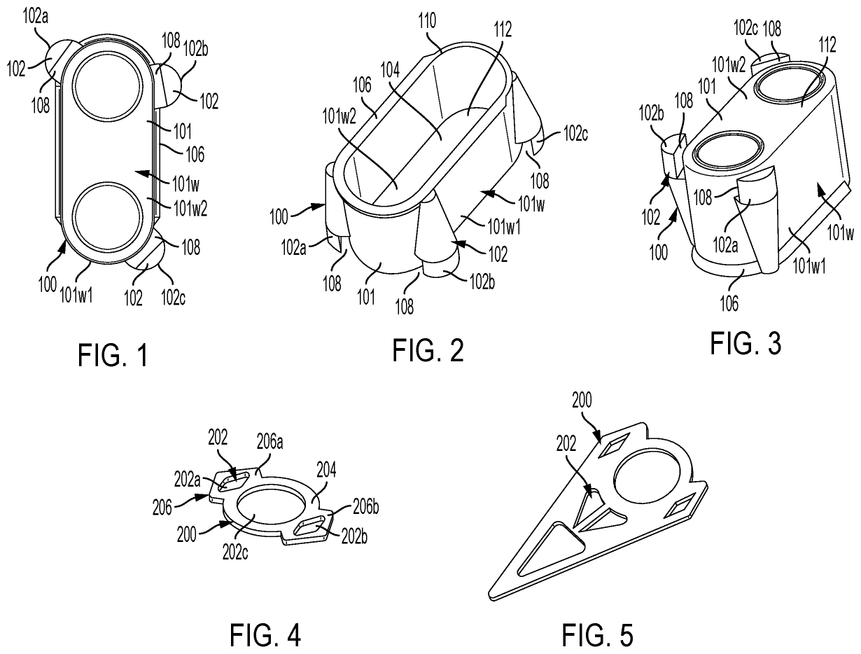

The features in FIGS. 1-23C can be combined with each other in any combination.

FIGS. 1-3 illustrate a variation of a diverter 100 (also referred to as a flow diverter 100). The diverter 100 can be removably attached to (e.g., removably positioned over) any device (e.g., any medical device) that ejects gas into the surrounding air, for example, to divert the flow of gas ejected from the device, to prevent the aerosolization of gas ejected from the device, or to divert the flow of gas ejected from the device and to prevent the aerosolization of gas ejected from the device. For example, the diverter 100 can removably cover the buttons of an endoscope to divert the flow of gas ejected from one of the buttons and/or to prevent the aerosolization of gas ejected from one of the buttons. An endoscope can be any device with a camera on it to look into anatomical areas of the human body.

The diverter 100 can be a flow diverter, can be an aerosolization preventer, or can be a flow diverter and an aerosolization preventer. The diverter 100 can divert the flow of gas ejected from a device to one or multiple locations, for example, to one or multiple exit ports on and/or within the diverter 100. For example, when the diverter 100 is removably secured to a device, a flow of gas from the device can be diverted via the diverter 100. The diverter 100 can prevent the aerosolization of gas from a device by collecting gas ejected from the device 100 and/or by diverting the flow of gas ejected from the device. For example, when the diverter 100 is removably secured to a device, aerosolization of gas ejected from the device can be prevented via the diverter 100.

FIGS. 1-3 illustrate that the diverter 100 can be, for example, a cap having a body 101, connectors 102, a cavity 104, and a lip 106, or any combination thereof.

The body 101 can have walls 101w, for example, a first wall 101w1 and a second wall 101w2. The first wall 101w1 can be attached to or integrated with the second wall 101w2. For example, FIGS. 1-3 illustrate that the first wall 101w1 can be integrated with the second wall 101w2. For example, the first wall 101w1 can be integrally formed with the second wall 101w2, for example, during a molding or 3D printing process. The first wall 101w1 can be, for example, a side wall that forms the perimeter of the cavity 104. The second wall 101w2 can be, for example, a central wall that extends across the cavity 104 to form an end (e.g., an apex) of the cavity 104. FIGS. 1-3 illustrate that the first wall 101w1 can have, for example, two straight sections and a two curved sections. The straight sections can form the lateral sides of the diverter 100, and the curved sections can form the longitudinal ends of the diverter 100 or vice versa.

The diverter 100 can have, for example, 0-6 connectors 102, including every 1 unit increment within this range (e.g., 0 connectors, 1 connector, 2 connectors, 6 connectors). For example, FIGS. 1-3 illustrate that the diverter 100 can have three connectors 102. FIGS. 1-3 illustrate, for example, that the connectors 102 can include a first connector 102a, a second connector 102b, and a third connector 102c. The connectors 102 can be integrated with or attached to the body 101. For example, FIGS. 1-3 illustrate that the connectors 102 can be integrally formed with the body 101, for example, during a molding or 3D printing process. The connectors 102 can be integrated with or attached to the first wall 101w1 and/or to the second wall 101w2. For example, FIG. 1-2 illustrate that the connectors 102 can be integrally formed with the first wall 101w1. The connectors 102 can extend from the body 101. The connectors 102 can extend from the first wall 101w1 and/or from the second wall 101w2. For example, FIGS. 1-3 illustrate that the connectors 102 can extend from the first wall 101w1.

FIGS. 1-3 illustrate that the connectors 102 can have the relative positions shown. For example, the first connector 102a and the second connector 102b can be on a first end of the diverter 100 and the third connector 102c can be on a second end of the diverter 100. The first end of the diverter 100 can be opposite the second end of the diverter 100. The first end of the diverter 100 can be a first longitudinal end of the diverter 100, and the second end of the diverter 100 can be a second longitudinal end of the diverter 100. The second connector 102b can be between first connector 102a and the third connector 102c. The connectors 102 can be positioned anywhere along the perimeter of the diverter 100. For example, FIGS. 1-3 illustrate that the first connector 102a can be on a first corner of the diverter 100, that the second connector 102b can be on a second corner of the diverter 100, and that the third connector 102c can be on a third corner of the diverter 100. FIG. 103 illustrate, for example, that the first and third connectors 102a, 102c can be on opposite corners of the diverter 100. FIGS. 1-3 illustrate that the second connector 102b can be, for example, on the second corner of the diverter 100 and on a straight section of the diverter 100 between the second and third corners of the diverter 100. FIGS. 1-3 illustrate that the fourth corner of the diverter 100 (e.g., the corner of the diverter 100 diagonally opposite the second corner of the diverter 100) may not have a connector 102. As another example, the fourth corner of the diverter 100 can have a connector 102.

FIGS. 1-3 illustrate that the cavity 104 can have the shape shown. The cavity 104 can have any shape, including, for example, the slot shape shown in FIGS. 1-3. As additional examples, the cavity 104 can have a circular shape, a triangular shape, a square shape, a rectangular shape, a diamond shape, or any other shape.

Each connector 102 can have 0-4 grooves 108, including every 1 groove increment within this range (e.g., 0 grooves, 1 groove, 4 grooves). One or multiple straps (e.g., one or multiple straps 200) can be inserted into the grooves 108. For example, FIGS. 1-3 illustrate that each of the connectors 102 can have one groove 108. A strap can extend through a groove 108, for example, when the diverter 100 is attached to a device with the strap. The grooves 108 can inhibit or prevent the straps from slipping off of the diverter 100.

The connectors 102 can be, for example, tether points (also referred to as connection points) that one or multiple straps (e.g., one or multiple straps 200) can releasably engage with to releasably secure the diverter 100 to a device. FIGS. 1-3 illustrate that the connectors 102 can be, for example, hooks that that one or multiple straps can engage with. For example, the connectors 102 and the grooves 108 can form hooks on the side of the diverter 100 that the straps can be removably attached to. FIGS. 1-3 illustrate that the connectors 102 can have the shapes shown. For example, FIGS. 1-3 illustrate that the connectors 102 can be posts with one or multiple grooves 108. FIGS. 1-3 illustrate, for example, that the connectors 102 can be tapered posts with a groove 108 on one of the ends. The connectors 102 can add rigidity to the diverter 100 while allowing the diverter 100 to retain enough flexibility so that a user can press buttons when the buttons are in the cavity 104.

The diverter 100 can have, for example, 0-4 cavities 104, including every 1 unit increment within this range (e.g., 1 cavity, 2 cavities, 4 cavities). For example, FIGS. 1-3 illustrate that the diverter 100 can have one cavity 104. FIGS. 1-3 that the cavity 104 can have an opening 110, for example, so that the diverter 100 can be positioned over a device (e.g., over buttons of an endoscope). The diverter 100 can have an opening 110 that can be, for example, the opening of the cavity 104. The opening 110 can be the entrance and/or exit to the cavity 104.

FIGS. 1-3 illustrate that the lip 106 can be at the base of the diverter 100. The lip 106 can removably engage with a device 100, for example, by being pressed into the device. The lip 106 can be, for example, a seal. The lip 106 can, for example, removably engage with a device with a friction fit. As another example, the lip 106 can removably engage with a groove on a device via a snap fit. The lip 106 can extend partially around or completely around the perimeter of the base of the diverter 100. For example, FIGS. 1-3 illustrate that the lip 106 can extend partially around the base of the diverter 100. FIGS. 1-3 illustrate, for example, that the first longitudinal end of the diverter 100 (e.g., the end having the first and second connectors 102a, 102b) can have the lip 106, and that the second longitudinal end of the diverter 100 (e.g., the end having the third connector 102c) may not have the lip 106. As another example, the lip 106 can extend completely around the base of the diverter 100.

FIG. 2 illustrates that a bottom surface of the diverter 100 and/or an inner surface of the cavity 104 can removably engage with a device, for example, to form a releasable seal against a device. FIG. 2 illustrates that the bottom surface of the diverter 100 can, for example, include a bottom surface of the cap and/or of the lip 106. When the diverter 100 is removably attached to a device, the bottom surface of the diverter 100 and/or the inner surface of the diverter 100 that defines the cavity 104 can have a friction fit against the device.

FIGS. 1-3 illustrate that the diverter 100 can be made of silicone. The diverter 100 can be transparent or opaque. FIGS. 1-3 illustrate that the diverter 100 can have a section 112. The section 112 can be opposite the opening 110. The section 112 can be, for example, the top of the diverter 100. The section 112 can be, for example, the second wall 101w2. When the diverter 100 is releasably attached to a device, the outer surface of section 112 can face away from the device. The section 112 can have a uniform thickness. As another example, FIGS. 1-3 illustrate that the outer surface can have two grooves (e.g., two circular grooves) to mark the positions of two buttons that can be positioned in the cavity 104 and/or to make it easier for the user to press buttons in the cavity 104 by increasing the flexibility of the section 110 in these regions.

FIG. 4 illustrates a variation of a strap 200 that can releasably secure the diverter 100 to a device. The strap 200 can have the features and shape shown. The strap 200 can have 0-6 openings 202, including every 1 opening increment within this range (e.g., 0 openings, 1 opening, 2 openings, 6 openings. For example, FIG. 4 illustrates that the strap 200 can have three openings 202, including, for example, a first opening 202a, a second opening 202b, and a third opening 202c, or any combination thereof. The first and second openings 202a, 202b can be on opposite ends of the strap 200. For example, FIG. 4 illustrates that the first opening 202a can be on a first longitudinal end of the strap 200, and that the second opening 202b can be on second longitudinal end of the strap 200. The first longitudinal end of the strap 200 can be opposite the second longitudinal end of the strap. FIG. 4 illustrates that the first opening 202a can have the same size and the same shape as the second opening 202b. As another example, the first opening 202a can have a different size and/or a different shape as the second opening 202b. FIG. 4 illustrates that the third opening 202c can be between the first opening 202a and the second opening 202b. FIG. 4 illustrates that the third opening 202c can be larger than the first opening 202a, and that the third opening 202c can be larger than the second opening 202b. The openings 202 can have any shape, including, for example, a circular shape, a triangular shape, a square shape, a rectangular shape, a diamond shape, or any other shape. For example, FIG. 4 illustrates that the first and second openings 202a, 202b can each have a diamond shape, and that the third opening 202c can have a circular shape. FIG. 4 illustrates that the first opening 202a can extend through the first longitudinal end of the strap 200, that the second opening 202b can extend through the second longitudinal end of the strap 200, and that the third opening 202c can extend through a longitudinal center of the strap 200. One or more openings 202 can extend partially through the strap 200, and/or one or more openings 202 can extend completely through the strap 200. For example, FIG. 4 illustrates that the openings 202 can extend completely through the strap 200 such that the openings 202 can be through holes. As another example, the openings 202 can extend partially through the strap 200 such that the openings 202 can be depressions or recesses on the surface of the strap 202. The openings 202 can be fit over the diverter 100, can be fit over the device that the diverter 100 is attached to, or both.

FIG. 4 illustrates that the strap 200 can be, for example, a loop 204 with 0-6 extensions 206, including every 1 extension increment within this range (e.g., 0 extensions, 1 extension, 6 extensions). The extensions 204 can extend from the loop 204. For example, FIG. 4 illustrates that two extensions 206 can extend from the loop 204, including, for example, a first extension 206a and a second extension 206b. The extensions 206 can be tabs that the user can pull on. For example, the extensions 206 can be grip tabs. As another example, the loop 204 may not have any extensions 206 such that the strap 200 can be a simple loop (e.g., a rubber band) has a single loop 104 with a single opening 202. FIG. 4 illustrates that the first opening 202a can extend through the first extension 206a, and that the second opening 202b can extend through the second extension 206b. FIG. 4 illustrates that the first extension 206a can be the first longitudinal end of the strap 200, and that the second extension 206b can be the second longitudinal end of the strap 200. The loop 204 can have any shape, including, for example, a circular shape, a triangular shape, a square shape, a rectangular shape, a diamond shape, or any other shape. For example, FIG. 4 illustrates that the loop 204 can have a circular shape. The extensions 206 can be attached to or integrated with the loop 204. For example, FIG. 4 illustrates that the strap 200 can be a unitary piece of material with three openings 206. As another example, the strap 200 may not have any openings 202, may not have any loops 204, and/or may not have any extensions.

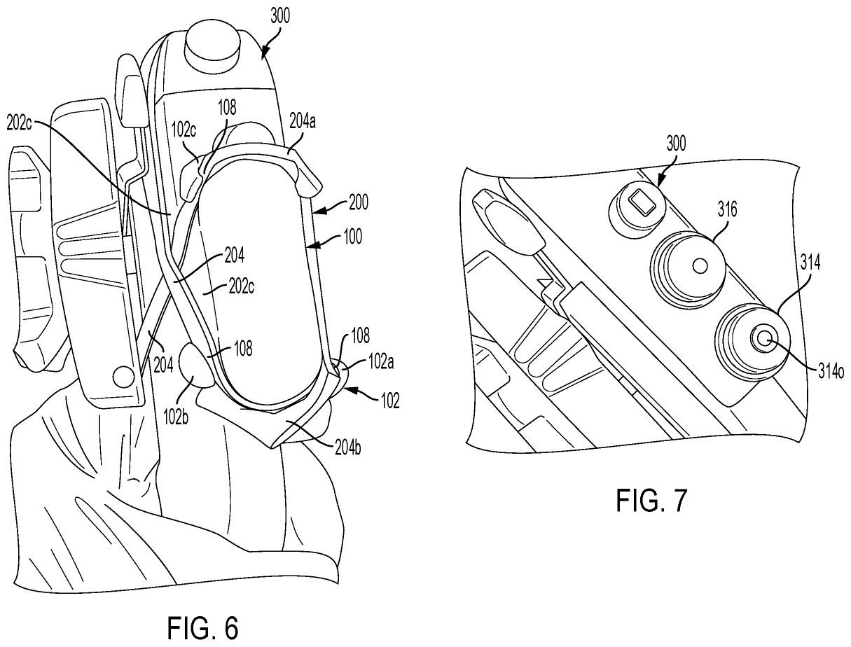

FIG. 5 illustrates that the strap 200 can have the features and shape shown. For example, FIG. 5 illustrates that the sealing strap 200 can have six openings 202 having the shapes, relative positions, and/or relative sizes shown.

The strap 200 can be elastic and/or inelastic. For example, FIGS. 4 and 5 illustrate that the strap 200 can be elastic. The strap 200 can be, for example, an elastic band having the openings 202. The strap 200 can be, for example, a silicone strap. The strap 200 can be, for example, a silicone band. As another example, the strap 200 can be, for example, a string that can be releasably tied to a device and/or to the diverter 100.

The diverter 100 can be removably attached (e.g., releasably tethered) to a device (e.g., an endoscope) to form a gas-tight (e.g., an air-tight) seal with zero, one, or two or more straps 200 depending on the situation. For example, FIG. 6 illustrates that the diverter 100 can be removably attached to a device 300 (e.g., to an endoscope 300) with two straps 200 to form a gas-tight seal between the diverter 100 and the device (e.g., the endoscope 300), including, for example, a first strap 200a and a second strap 200b. FIG. 6 illustrates that the first strap 200a can be the same as the strap 200 shown in FIG. 4, and that the second strap 200b can be the same as the strap 200 shown in FIG. 4. FIG. 6 illustrates that the second strap 200b can extend over the first strap 200a or vice versa. For example, FIG. 6 illustrates that the loop 204 of the second strap 200b can extend over the loop 204 of the first strap 200a or vice versa. As another example, more than two straps 200 can be used, for example, 3-5 straps 200. When zero straps 200 are used, the diverter 100 can be left loose on the device. In such cases, the diverter 100 can be removably attached to the device, for example, via friction fit between the base of the diverter 100 and the device.

When the diverter 100 is removably secured to a device (e.g., to the endoscope 300), the diverter 100 can extend through an opening 202 of the first strap 202a and/or the diverter 100 can extend through an opening 202 of the second strap 202b. For example, FIG. 6 illustrates that when the diverter 100 is removably secured to a device (e.g., to the endoscope 300), the diverter 100 can extend through the third opening 202c of the first strap 202a and the diverter 100 can extend through the third opening 202c of the second strap 202b.

When the diverter 100 is removably secured to a device (e.g., to the endoscope 300), the device (e.g., the endoscope 300) can extend through an opening 202 of the first strap 202a and/or the device (e.g., the endoscope 300) can extend through an opening 202 of the second strap 202b. For example, FIG. 6 illustrates that when the diverter 100 is removably secured to a device (e.g., to the endoscope 300), the device (e.g., the endoscope 300) can extend through the third opening 202c of the first strap 202a and/or the device (e.g., the endoscope 300) can extend through the third opening 202c of the second strap 202b.

FIG. 6 illustrates that the diverter 100 can cover buttons of the endoscope 300. FIG. 6 illustrates, for example, that buttons of the endoscope 300 can be in the cavity 104 of the diverter 100. FIG. 6 illustrates that the diverter 100 can be placed over the buttons of the endoscope 300 and that the strap 200 can be placed over the endoscope 300 onto the connectors 102 (e.g., tether points) to removably secure the diverter 100 to the endoscope 300 with a gas-tight seal (e.g., an air-tight seal).

FIGS. 4-6 illustrate that the strap 200 can have a non-stretched configuration (e.g., as shown in FIGS. 4 and 5) and that the strap 200 can have a stretched configuration (e.g., as shown in FIG. 6). FIGS. 4-6 illustrate that the strap 200 can be stretched to attach the diverter 100 to the endoscope 300.

FIG. 6 illustrates that the diverter 100 can be removably attached to the handle of the endoscope 300.

FIG. 6 illustrates that the diverter 100 can assist with the gripping of the buttons in case of aiding the operator to use the buttons of the endoscope 300.

The diverter 100 and the strap 200 can be flexible and can be composed of a variety of different materials. For example, the diverter 100 can be made of polyurethane of a variety of different durometers and/or of silicone, and the strap 200 can be made of, for example, silicone. The material composition of the diverter 100 and/or of the one or multiple straps 200 can be changed in order to suit the sealing degree needed for the clinical application. The diverter 100 can be flexible and/or elastic. The strap 200 can be flexible and/or elastic.

FIG. 6 illustrates that the diverter 100 can be opaque. As another example, the diverter 100 can be transparent.

Endoscopes typically have a gas/water button and a suction button. The diverter 100 can be placed over the gas/water button and/or over the suction button. For example, FIG. 6 illustrates the diverter 100 positioned over the gas/water button and the suction button of the endoscope 300.

FIG. 7 illustrates an arrangement of a gas/water button 314 and a suction button 316 that endoscopes (e.g., the endoscope 300) typically have. For example, FIG. 7 illustrates that the endoscope 300 can have the gas/water button 314 and the suction button 316. The gas/water button 314 can be, for example, an air/water button or a carbon dioxide/water button. For endoscopes that have a water control button that is separate from the gas control button, the gas/water button 314 can be a gas button and another control on the endoscope can be the water button. The gas/water button 314 can also be referred to as a gas/water valve. The suction button 316 can also be referred to as a suction valve.

FIG. 7 illustrates that the gas/water button 314 can have an opening 314o that gas (e.g., air, carbon dioxide) can exit from during operation of the endoscope 300. The user can control the gas flow and water flow through the endoscope 300 with the gas/water button 314 and can control the suction through the endoscope 300 with the suction button 316. During operation, the endoscope 300 can be connected to a pump (e.g., an air pump, a carbon dioxide pump), a water source (e.g., a water bottle), and a suction source. When the gas pump is switched on and the opening 314o is uncovered, gas flows out (e.g., hisses out) of the gas/water button 314 through the opening 314o. Silence from the opening 314o can inform the user that the pump is not powered on. Covering the opening 314o (e.g., with a finger) will blow air through the tip of the endoscope 300 and inflate the anatomy that the tip of the endoscope 300 is in (e.g., the small intestine, the large intestine, the stomach). Pressing the gas/water button (e.g., pressing the gas/water button fully down) washes the lens of the endoscope 300 with water, for example, from a water bottle. When the suction button 316 is pressed, water/gas is sucked into through the tip of the endoscope 300. The suction channel is also connected to the working channel (e.g., accessory/biopsy channel).

To inflate a target space (e.g., the small intestine, the large intestine, the stomach) with current endoscopes (e.g., represented by endoscope 300), the user can cover the opening 314o on the gas/water button 314 to direct the flow of gas (e.g., air, carbon dioxide) to the distal end of the endoscope 300 to inflate anatomy (e.g., the small intestine, the large intestine, the stomach). When the opening 314o is covered, gas is blocked from exiting the opening 314o and is forced into the anatomy that the tip of the endoscope 300 is in (e.g., the small intestine, the large intestine, the stomach). When the opening 314o of the gas/water button is not covered, the gas that would otherwise flow into the patient when the opening 314o is covered flows out of the gas/water button 314 through the opening 314o. This design, however, can lead to gas and/or particles from inside the patient being ejected (e.g., aerosolized) from the opening 314o into the space (e.g., the room) the endoscope 300 and the patient are in after the user inflates the target space and then uncovers the opening 314o. This is because once the user inflates the target space and uncovers the opening 314o, the gas that flows out of the opening 314o can be gas from the pump that is connected to the endoscope 300 and can be gas that was forced into the target space. In other words, once the user inflates the target space and uncovers the opening 314o, the gas exiting the opening 314o can be a mix of the gas (e.g., air, carbon dioxide) from the air pump and of gas and particles inside the target space. This is undesirable from an infection control standpoint because such particles that flow out of the opening 314o (e.g., that are aerosolized by flowing through the opening 314o) from inside the patient can be infectious. For example, when the gastrointestinal (GI) tract is inflated with an endoscope (e.g., the endoscope 300), such particles can be coronavirus (COVID) particles, including, for example, severe acute respiratory syndrome coronavirus 2 (SARS-CoV-2) particles, which have been shown to be concentrated in the gut. It is therefore highly desirable to prevent the gas and/or particles from inside the patient (e.g., from the target space, from the inflated target space) from being aerosolized in the space (e.g., room) that the user and the patient are in, to contain the gas and/or particles from inside the patent that flow out of the opening 314o, and/or to divert the flow of the gas and/or particles from inside the patient that flow out of the opening 314o away from the user and/or away from the user's team members.

To address this problem of ejecting gas (e.g., air, carbon dioxide) from the opening 314o that can be a mix of gas from the patient that can have infectious particles (e.g., including COVID particles), the diverter 100 can be attached to the endoscope (e.g., to the endoscope 300) to cover the gas/water button (e.g., the gas/water button 314) or to cover the gas/water button and the suction button (e.g., the gas/water button 314 and the suction button 316).

The diverter 100 can be attached to the endoscope 300 (e.g., as shown in FIG. 6) to divert the flow of gas ejected from the endoscope 300 (e.g., from the gas/water button 314), to prevent the aerosolization of gas ejected from the endoscope 300 (e.g., from the gas/water button 314), or to divert the flow of gas ejected from the endoscope 300 (e.g., from the gas/water button 314) and to prevent the aerosolization of gas ejected from the endoscope 300 (e.g., from the gas/water button 314). For example, the diverter 100 can removably cover the gas/water button 314 or the gas/water button 314 and the suction button 316 to divert the flow of gas ejected from the gas/water button 314 and/or to prevent the aerosolization of gas ejected from the gas/water button 314. For example, the diverter 100 can be positioned over the gas/water button 314 or over the gas/water button 314 and the suction button 316 of the endoscope 300 to divert the flow of gas from the gas/water button 314 (e.g., from the opening 314o) and/or to prevent the aerosolization of gas from the gas/water button 314 (e.g., from the opening 314o).

When the diverter 100 is attached to an endoscope (e.g., the endoscope 300), the diverter 100 can divert the flow gas and/or particles ejected from the opening 314o in a different direction (e.g., away from the user), can prevent the flow gas and/or particles ejected from the opening 314o from being released into the space (e.g., room) that the endoscope 300 is in, can move the point at which the gas and/or particles ejected from the opening 314o are released into the space (e.g., the room) that the endoscope 300 is in, can route the flow gas and/or particles ejected from the opening 314o to a filter, or any combination thereof. The diverter 100 can, for example, prevent the aerosolization of infectious particles (e.g., COVID particles). During use, the opening 314o typically faces the user and their face. The diverter 100 can, for example, prevent the endoscope 300 from blowing infectious particles into the user through the opening 314o.

FIG. 6 illustrates, for example, that the diverter 100 can be positioned over the gas/water button 314 and the suction button 316 of an endoscope. FIG. 6 illustrates, for example, that that the diverter 100 can cover the gas/water button 314 and the suction button 316 of the endoscope 300. The gas/water button 314 or the gas/water button 314 and the suction button 316 can be positioned in the cavity 104 of the diverter 100. FIG. 6 illustrates, for example, that the gas/water button 314 and the suction button 316 of the endoscope 300 can be positioned in the cavity 104 of the diverter 100. FIG. 6 illustrates, for example, that the diverter 100 can be placed over the gas/water button 314 and the suction button 316 and that straps 200 can be placed over the endoscope 300 and the diverter 100 (e.g., onto the connectors 102) to removably secure the diverter 100 to the endoscope 300 with a gas-tight seal (e.g., an air-tight seal). As another example, the diverter 100 can be placed over the gas/water button 314 and the suction button 316 and that straps 200 can be placed over the endoscope 300 and the diverter 100 (e.g., onto the connectors 102) to removably secure the diverter 100 to the endoscope 300 without a gas-tight seal (e.g., an air-tight seal). The diverter 100 can prevent the flow of gas and/or particles that flow from the opening 314o from blowing directly onto the operator (also referred to as the user). When the opening 314o is uncovered, the flow of gas from the opening 314o can be, for example, 3 inches to 12 inches in length. The diverter 100 can contain this flow of gas and/or modify this flow of gas, for example, by modifying the shape of the flow and/or by splitting the flow into multiple flows.

When the diverter 100 is removably secured to the endoscope 300 (e.g., with one or more straps 200), the diverter 100 can be sealed against the endoscope 300 with a gas-tight seal (e.g., an air-tight seal). The gas-tight seal can be a gas-tight suction seal (e.g., an air-tight suction seal, a carbon dioxide-tight suction seal). A gas-tight seal between the base of the diverter 100 and the endoscope 300 (e.g., the handle of the endoscope 300) can prevent the flow of gas and/or particles that flow from the opening 314o from exiting the cavity 104 between the base of the diverter 100 and the endoscope 300 (e.g., the handle of the endoscope 300). In such cases, the diverter 100 can direct the gas and/or particles that flow into the cavity 104 from the opening 314o into the suction button 314 and/or into a luer, for example, to direct the flow of gas and/or particles that flow from the opening 314o in a direction toward the user into a direction away from the user and/or to direct the flow of gas and/or particles that flow from the opening 314o to a filter that can, for example, filter (e.g., scrub) the particles (e.g., infectious particles) from the gas.

As another example, when the diverter 100 is removably secured to the endoscope 300 (e.g., with one or more straps 200), the diverter 100 may not be sealed against the endoscope 300 with a gas-tight seal (e.g., an air-tight seal, a carbon dioxide-tight seal), for example, so that the diverter 100 can direct a flow of gas out of the cavity 104 between the base of the diverter 100 and the endoscope 300 (e.g., between the diverter 100 and the handle of the endoscope 300) in one or multiple flow channels. In such cases, the gas and/or particles that flow out of the cavity 104 between the base of the diverter 100 and the handle of the endoscope 300 can flow away from the operator of the endoscope 300. The diverter 100 can thereby direct the flow of gas and/or particles that flow from the opening 314o in a direction toward the user into a direction away from the user.

FIG. 7 illustrates, for example, the endoscope 300 of FIG. 6 without the diverter 100. FIG. 7 illustrates, for example, the endoscope 300 before the diverter 100 is attached to the endoscope 300. FIG. 7 illustrates, for example, the endoscope 300 after the diverter 100 is detached from the endoscope 300.

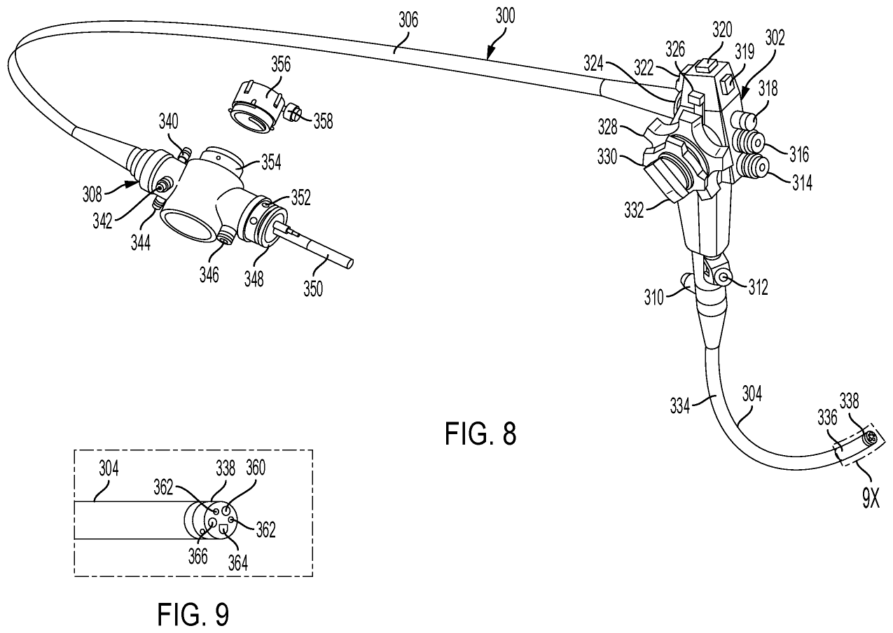

FIG. 8 illustrates an exemplary details of an endoscope, for example, of the endoscope 300 in FIG. 6. FIG. 8 illustrates that an endoscope (e.g., the endoscope 300) can have, for example, a control section 302 (e.g., a handle), an insertion tube 304, a tube 306, and a connector 308.

FIG. 8 illustrates that the control section 302 can have an auxiliary water inlet 310, a working channel valve 312 (e.g., a biopsy valve), the gas/water button 314, the suction button 316, a switch 318, a switch 319, a switch 320, a switch 322, a product series indicator 324, a lock 326 (e.g., an up/down angulation lock), a control 328 (e.g., an up/down angulation control knob), a control 330 (e.g., a right/left angulation control knob), and a lock 332 (e.g., a right/left angulation lock), or any combination thereof.

FIG. 8 illustrates that the insertion tube 304 can have a flexible portion 334, a deflectable section 336, and a distal end 338, or any combination thereof.

FIG. 8 illustrates that the connector 308 can have a suction connector 340, a gas tube connector 342, an S-Cord connector 344, a water source connector 346, a gas pipe 348 (e.g., an air pipe), a light guide 350, electrical contacts 352, an electrical connector 354, a water resistant cap 356 that can be removably connectable to the electrical connector 354, and a leak tester connector 358, or any combination thereof. The suction connector 340 can be connected to a scrubber that cleans the gas (e.g., air) in the suction channel 390.

FIG. 9 illustrates that the distal end 338 of the insertion tube 304 can have an objective lens 360, a light guide 362, a nozzle 364 (e.g., a gas/water nozzle), and an instrument channel 366, or any combination thereof.

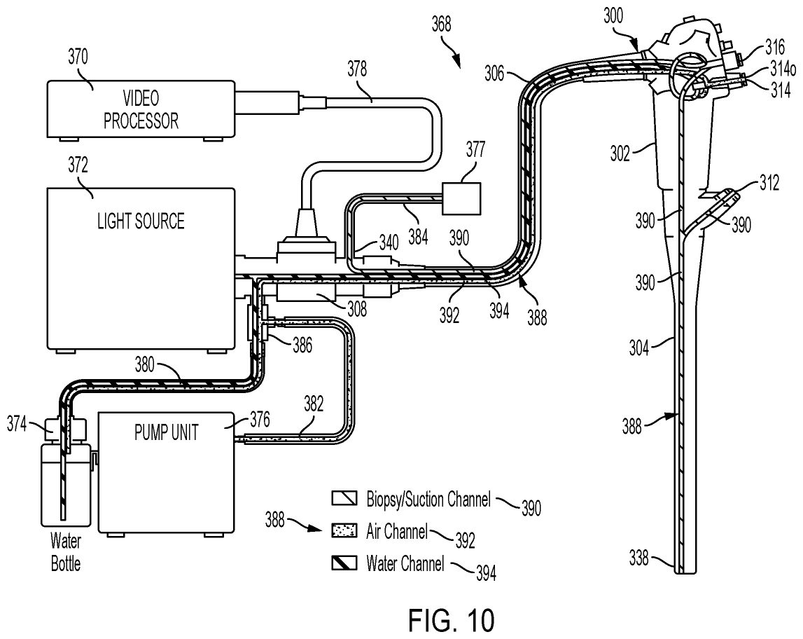

While the specific design of endoscopes differ across different manufacturers, endoscopes typically have a gas/water button and a suction button (e.g., the gas/water button 314 and the suction button 316). This disclosure is not limited to a single endoscope design. The diverter 100 can be, for example, removably attached to any medical device, including any endoscope, that releases a flow of gas and/or particles from the device. In other words, FIGS. 6-9 are not shown to limit the disclosure to single endoscope design but are instead shown to represent an exemplary medical device (e.g., an endoscope) that can release a flow of gas and/or particles that the diverter 100 can control. FIGS. 6 and 7 generally illustrate, for example, that this release of gas and/or particles can be through an opening in a button on a device, and that this button can be, for example, a gas button (e.g., the gas/water button 314) on an endoscope (e.g., the endoscope 300). The additional details in FIGS. 10-13B are shown and described below, for example, to assist in understanding how an endoscope (e.g., the endoscope 300) functions, including, for example, how the diverter 100 can function with the gas, water, and/or suction channels of an endoscope (e.g., the endoscope 300).

FIG. 10 illustrates the anatomy of an exemplary endoscopy system 368. FIG. 10 illustrates that the endoscopy system 368 can include the endoscope 300, a video processor 370, a light source 372, a water source 374, a pump 376 (e.g., an air pump, a carbon dioxide pump), and a suction source 377, or any combination thereof. FIG. 10 illustrates, for example, that the endoscope 300 can be connected to the video processor 370, to the light source 370, to the water source 374, to the pump 376, and to the suction source 277 via the connector 308. FIG. 10 illustrates, for example, that the endoscope 300 can be connected to the video processor 370 via an electrical cable 378, that the endoscope 300 can be connected to the light source 372 via the light guide 350 and the electrical contacts 352, that the endoscope 300 can be connected to the water source 374 via a water tube 380, that the endoscope 300 can be connected to the pump 376 via a gas tube 382 (e.g., an air tube), and that the endoscope 300 can be connected to the suction source 377 via a suction tube 384. A first end of the water tube 380 can be connected to the water source connector 346 and a second end of the water tube 380 can be connected to the water source 374. A first end of the gas tube 382 can be connected to the gas tube connector 342 and a second end of the gas tube 382 can be connected to the pump 376. A first end of the suction tube 384 can be connected to the suction connector 340 and a second end of the suction tube 384 can be connected to the suction source 377. FIG. 10 illustrates that the endoscopy system 368 can have a gas adapter 386.

FIG. 10 illustrates that the endoscopy system 368 can have a gas (e.g., air, carbon dioxide), water, and suction channel system 388 (also referred to as the channel system 388). FIG. 10 illustrates that the channel system 388 can include a suction channel 390, a gas channel 392, and a water channel 394.

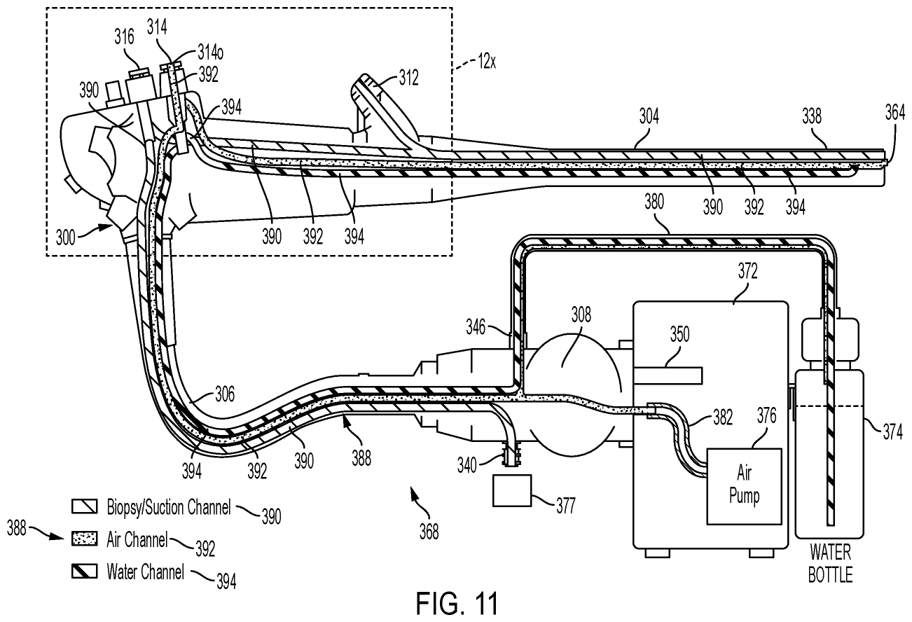

FIG. 11 illustrates a closeup of the endoscope 300 and a closeup of the channel system 388. FIG. 11 illustrates that the pump 376 can be housed in the light source 372.

FIGS. 10 and 11 illustrate that the suction channel 390 can extend from the suction source 377 through the endoscope 300 to the suction button 316, to the working channel valve 312, and to the distal end 338 of the endoscope 300. FIG. 10 illustrates that the portion of the suction channel 390 between the working channel valve 312 and the distal end 338 of the endoscope 300 can function as both a working channel (e.g., a biopsy channel) and as a suction channel. To reflect this dual functionality of this portion of the suction channel 390, the legends in FIGS. 10 and 11 refer to the suction channel 390 as a biopsy/suction channel 390.

FIGS. 10 and 11 illustrate that the gas channel 392 can extend from the water source 374 and from the pump 376 though the endoscope 300 to the gas/water button 314 and to the distal end 338 of the endoscope 300. In FIG. 10, for example, the gas channel 392 is shown behind the suction channel 390 in the control section 302 and in insertion tube 304 of the endoscope 300. The gas channel 392 can be any gas channel. Any gas can flow through the gas channel 392, including, for example, air, carbon dioxide, and/or gas from the patient. The pump 376 can be, for example, an air pump or a carbon dioxide pump. For example, the legends in FIGS. 10 and 11 refer to the gas channel 392 as an air channel 392. As another example, the gas channel in FIGS. 10 and 11 can be a carbon dioxide channel.

FIGS. 10 and 11 illustrate that the water channel 394 can extend from the water source 374 though the endoscope 300 to the gas/water button 314 and to the distal end 338 of the endoscope 300. In FIG. 10, for example, the water channel 394 is shown behind the suction channel 390 in the control section 302 and in insertion tube 304 of the endoscope 300.

FIGS. 10 and 11 illustrate that there are multiple channels within the handle of the endoscope 300 which circuits are selectively completed by covering the opening 314o, by pressing the gas/water button 314, and/or by pressing the suction button 316.

FIGS. 10 and 11 illustrate the endoscope 300, the water tube 380, the gas tube 382, and the suction tube 384 transparent so that the channel system 388 can be seen.

FIGS. 10 and 11 illustrate that once the user inflates a target space (e.g., the small intestine, the large intestine, the stomach) and uncovers the opening 314o, the gas that flows out of the opening 314o can be gas that flows through the gas channel 392 from the pump 376 and can be gas that flows through the gas channel 392 from target space. The gas in the target space can enter the endoscope 300 (e.g., can enter the gas channel 392) through the same opening that it flowed through to inflate the target space and/or through a different opening. For example, after inflation of the target space, when the opening 314o is uncovered, gas (e.g., air, carbon dioxide) from the target space can flow into the gas channel 392 through a distal opening in the insertion tube 104 (e.g., through the nozzle 364 and/or a working channel opening). In other words, once the user inflates the target space and uncovers the opening 314o, gas that exited the gas channel 392 can renter the gas channel 392. FIGS. 10 and 11 thereby show that once the user inflates the target space and uncovers the opening 314o, the gas exiting the opening 314o can be a mix of gas from the pump 376 and the target space, for example, of gas from the pump 376 and of gas and particles that entered the gas channel 392 from inside the target space.

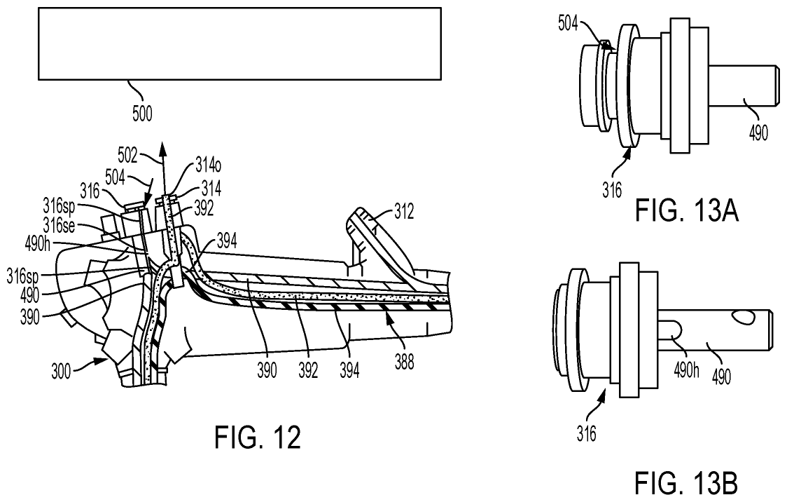

FIG. 12 illustrates that when the opening 314o is not covered, a flow 502 of gas and/or particles can flow from the opening 314o, for example, toward a user 500. The flow 502 can include gas from the pump (e.g., the pump 376) and/or can include gas that was forced into the target space. In other words, once the user inflates a target space and uncovers the opening 314o, the flow 502 exiting the opening 314o can be a mix of the gas (e.g., air, carbon dioxide) from the pump (e.g., the pump 376) and of gas and/or particles from the target space. FIG. 12 illustrates that during operation of the endoscope 300 without the diverter 100 attached to the endoscope 300, gas and/or particles can be ejected from the opening 314o in the gas/water button 314. This ejection of gas and/or particles is represented in FIG. 12 as the flow 502. As shown in FIG. 12, the flow 502 can be a flow of gas and/or particles out of the endoscope 300.

FIG. 12 illustrates that when the suction button 316 is not pressed, a flow 504 of gas and/or particles can flow into the suction button 316. The flow 504 can be caused, for example, by suction in the suction channel 390. For example, when the suction button 316 is not pressed, suction from the suction channel 390 can leak through the suction button 316, which can create the flow 504 into a gap around the perimeter of the suction button 316 that is in communication with the suction channel 390. For example, FIGS. 12 and 13A illustrate that when the suction button 316 is in a non-pressed configuration, a rod 490 can extend through the suction channel 390 to block suction from reaching the tip of the endoscope 300, and FIG. 13B illustrates that when the suction button 316 is in a pressed configuration (e.g., in a fully pressed configuration), a hole 490h in the rod 490 can be aligned with the suction channel 390 so that the tip of the endoscope 300 can deliver suction to the target site. FIGS. 12 and 13A illustrate, for example, that when the suction button 316 is in a non-pressed configuration, the hole 490h can be misaligned with the suction channel 390 to block suction from reaching the tip of the endoscope 300. However, when the suction button 316 is in a non-pressed state as shown in FIGS. 12 and 13A, a seal 316se between the rod 490 and the suction channel 390 may not be suction tight such that the seal 316se can leak suction, for example, into a gap in the perimeter of the suction button 316. The suction that leaks between the suction channel 390 and the rod 490 when the suction button 316 is not pressed can cause a flow (e.g., the flow 504) to be sucked into the suction channel 390 from outside the endoscope 300 through the suction button 316 (e.g., through a space that extends from the perimeter of the suction button 316) into the suction channel 390. For example, FIG. 12 illustrates that the flow 504 of gas and/or particles can flow into the suction channel 390 through a space 316sp that extends through the suction button 316 to the seal 316se, around or through the seal 316se, and into the suction channel 390 (e.g., into the portion of the suction channel 390 between the suction button 316 and the suction source 377). FIG. 12 illustrates that the space 316sp can be, for example, a channel that extends along the rod 490. FIG. 12 illustrates that the space 316sp can extend all the full length of the rod 490, whereby the gap in the space 316sp in FIG. 12 is illustrated so that the hole 490h and the seal 316se can be seen and to show that the space 316sp can extend around the seal 316se into the suction channel 390. As shown in FIGS. 12 and 13A, the flow 504 can be a flow of gas and/or particles into the endoscope 300, for example, into the suction channel 390 through a space (e.g., the space 316sp) that extends through suction button 316, for example, through a space (e.g., the space 316sp) that extends from an outer surface of the suction button 316 (e.g., from an edge or side of the suction button 316) to the suction channel 390. FIG. 13B illustrates that when the suction button 316 is in a pressed configuration, the space (e.g., the space 316sp) that extends from an outer surface of the suction button 316 to the suction channel 390 when the suction button 316 is not pressed can be closed such that pressing the suction button 316 can terminate the flow 504. FIGS. 12-13B illustrate that endoscopes (e.g., the endoscope 300) can leak suction through the suction button 316 when attached to suction (e.g., when attached to the suction source 377). As another example, the suction button 316 may not leak suction, in which case, the endoscope 300 may not have the flow 504.

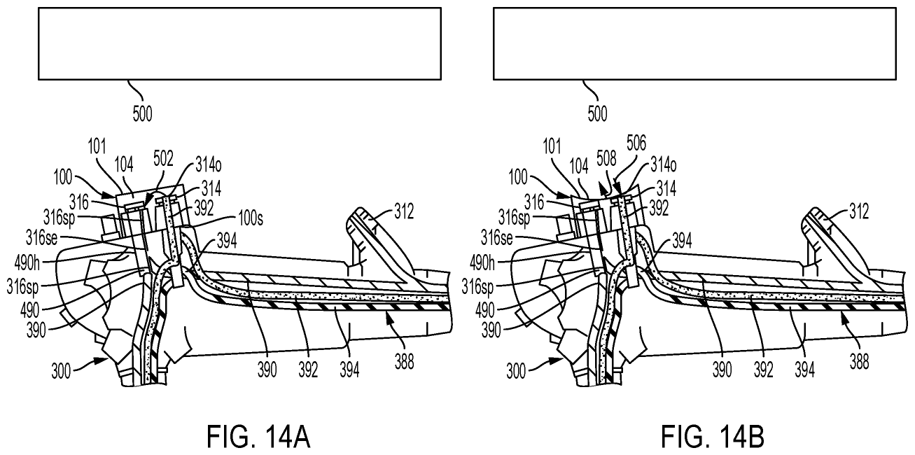

FIGS. 14A and 14B illustrate that the diverter 100 can be removably attached to the endoscope 300, for example, with a friction fit. FIGS. 14A and 14B illustrates that the diverter 100 can be removably attached to the endoscope without a strap 200. As another example, the diverter 100 can be attached to the endoscope 300 with one or more straps 200. FIGS. 14A and 14B illustrates that the diverter 100 may not have any connectors 102. As another example, the diverter 100 can have connectors 102, for example, the connectors 102 shown in FIGS. 1-3. FIGS. 14A and 14B illustrate that the diverter 100 can be transparent.

FIG. 14A illustrates that when the opening 314o is open (e.g., not covered) and when the suction button is in a non-pressed configuration, the flow 502 of gas and/or particles from the gas channel 392 can flow out the opening 314o, into the cavity 104, and into the suction channel 390 through the suction button 316 (e.g., through the space 316sp). FIG. 14A illustrates that when the diverter 100 is attached to the endoscope 300, the diverter 100 can cover but not occlude the opening 314o. This can allow the flow 502 of gas and/or particles from the gas channel 392 to flow out of the opening 314o and into the suction channel 390 through the suction button 316. This can allow the flow 502 of gas and/or particles from the gas channel 392 to flow out of the opening 314o, accumulate in the cavity 104, and then flow into the suction channel 390 through the suction button 316.

FIG. 14B illustrates that the user 500 can close the opening 314o by pressing the diverter 100 (e.g., the top of the diverter 100, a wall 101w of the diverter 100) into contact with the gas/water button 314 to occlude the opening 314o as shown by arrow 506, for example, via a finger. FIG. 14B illustrates that the top of the diverter 100 can be, for example, the second wall 101w2. When the opening 314o is closed by occluding the opening 314o with the diverter 100, the flow 502 of gas and/or particles from the gas channel 392 out the opening 314o can be terminated as shown in FIG. 14B. Once the user releases the diverter 100, the diverter 100 can automatically return (e.g., rebound) to the shape shown in FIG. 14A, for example, as shown by arrow 508. When the user opens (e.g., reopens) the opening 314o by releasing the diverter 100 as shown by arrow 508, the flow 502 of gas and/or particles from the gas channel 392 can flow (e.g., can once again flow) out the opening 314o, into the cavity 104, and into the suction channel 390 through the suction button 316 (e.g., through the space 316sp) as shown in FIG. 14A. As another example, FIGS. 14A and 14B illustrate that the user can press and depress the gas/water button 314 and/or the suction button 316 as shown by arrows 506 and 508, respectively. When the user presses and depresses the gas/water button 314, the diverter 100 can move, for example, in directions 506 and 508, respectively. Direction 506 can be a first direction and direction 508 can be a second direction opposite the first direction (e.g., direction 506 can be opposite direction 508). When the user presses and depresses the suction button 316, the diverter 100 can move back and forth, for example, in directions 506 and 508, respectively. FIGS. 14A and 14B thereby illustrate that the user can use the gas/water button 314 while the diverter 100 is attached to the endoscope 300.

FIGS. 14A and 14B illustrate that when the diverter 100 is removably attached to the endoscope 300, the base of the diverter 100 can be in contact with the endoscope 300 (e.g., with a surface of the handle of the endoscope 300) such that a seal 100s is formed between the base of the diverter 100 and the endoscope 300. The seal 100s can be a gas-tight seal that can prevent gas and/or particles from escaping the cavity 104 between the base of the diverter 100 and the endoscope 300. In such cases, diverter 100 can divert the flow 502 of gas and/or particles from the gas channel 392 back into the endoscope 300 via a space (e.g., the space 316sp) that extends from an outer surface of the button to the suction channel 390. As another example, the seal 100s may not be a gas-tight seal such that gas and/or particles can escape the cavity 104 through both the suction button 316 and through the seal 100s (e.g., through a space between the bottom surface of the diverter 100 and the surface of the endoscope 300).

FIGS. 14A and 14B illustrate that the diverter 100 can be deflected toward the gas/water button 314 to block the opening 314o with the diverter 100 (e.g., as shown by arrow 506), and illustrate that the diverter 100 can be deflected away from the gas/water button 314 to unblock the opening 314o of the diverter 100 (e.g., as shown by arrow 508). FIGS. 14A and 14B thereby illustrate that the diverter 100 can be deflected into and out of contact with the gas/water button 314 to close and open the opening 314o, respectively.

FIGS. 14A and 14B illustrate that the diverter 100 can be deflected to block and unblock the opening 314o in directions 506 and 508, respectively, without breaking the seal 100s.

FIGS. 14A and 14B illustrate that the user can press and depress the gas/water button 314 and/or the suction button 316 while the diverter 100 is attached to the endoscope 300 without breaking the seal 110s.

FIGS. 14A and 14B illustrate that the diverter 100 can have a non-deflected configuration (e.g., as shown in FIG. 14A) and a deflected configuration (e.g., as shown in FIG. 14B. FIG. 14A illustrates that when the diverter 100 is in a non-deflected configuration, a gap 100g can be between the opening 314o and the top of the diverter 100 (e.g., the second wall 101w2 of the diverter 100). FIG. 14A illustrates that when the diverter 100 is in a non-deflected configuration, the gap 100g can be between the gas/water button 314 and the top of the diverter 100 (e.g., the second wall 101w2 of the diverter 100). FIG. 14A illustrates that when the diverter 100 is in a non-deflected configuration, the gap 100g can be between the gas/water button 314 and the top of the diverter 100 (e.g., the second wall 101w2 of the diverter 100). The gap 100g can be for example, 2 mm to 10 mm, including every 1 mm increment within this range (e.g., 2 mm, 3 mm, 5 mm, 10 mm) For example, FIG. 14A illustrates that the gap 100 can be 3 mm or 4 mm. The gap 100g can allow the flow 502 of gas and/or particles from the opening 314o to flow into the cavity 104. The gap 100g (e.g., height difference between the body 101 and the opening 314o, the gas/water button 314, and/or the suction button) does not cause occlusion when the diverter 10 is not pressed to occlude the opening 314o or to press the either of the buttons.

When the diverter 100 is in the non-deflected configuration, the top of the diverter 100 (e.g., the second wall 101w2) can be movable (e.g., deflectable) toward the opening 314o, toward the gas/water button 314, and/or toward the suction button 316, for example, in direction 506. FIG. 14B illustrates that when the diverter 100 is in a deflected configuration, diverter 100 can occlude the opening 314o, can contact the gas/water button 314, and/or can contact the suction button 316 such that the diverter 100 may not have the gap 100g. When the diverter 100 is in the non-deflected configuration, the top of the diverter 100 (e.g., the second wall 101w2) can be movable (e.g., deflectable) away the opening 314o, away from the gas/water button 314, and/or away the suction button 316, for example, in direction 508. The deflected configuration in FIG. 14B can be, for example, a partially defected configuration or a fully deflected configuration of the diverter 100.

FIGS. 14A and 14B illustrate that the diverter 100 can removably cover the gas/water button 314 and the suction button 316 to divert the flow of gas and/or particles ejected from opening 314o away from the user 500, for example, into the suction button 316.

FIGS. 14A and 14B illustrate that the diverter 100 can prevent the aerosolization of gas and/or particles ejected from the opening 314o outside of the cavity 104.

FIGS. 14A and 14B illustrate that when the diverter 100 is attached to an endoscope (e.g., the endoscope 300), the diverter 100 can divert the flow gas and/or particles ejected from the opening 314o in a different direction (e.g., away from the user) and can prevent the flow gas and/or particles ejected from the opening 314o from being released into the space (e.g., room) that the endoscope 300 is in.

FIGS. 14A and 14B illustrate that the flow 502 of gas and/or particles can flow out of the cavity 104, for example, through the suction button 316. The flow 502 of gas and/or particles can, for example, then be suctioned to the suction source and/or to a filter.

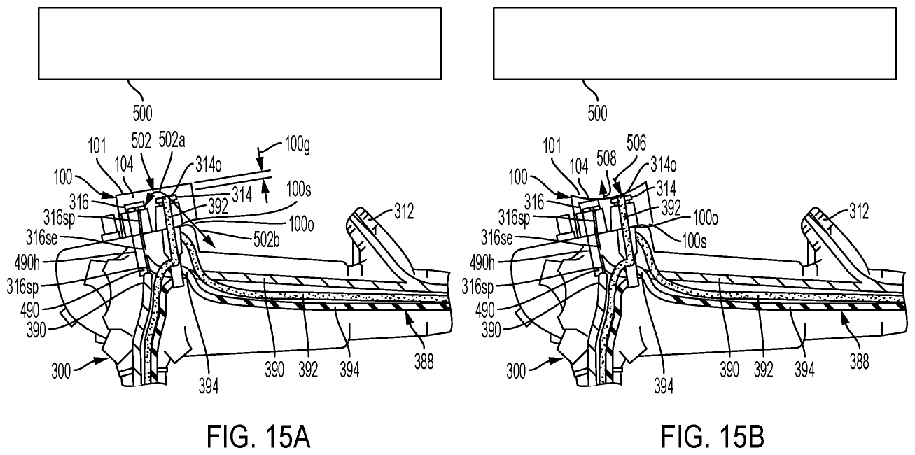

FIGS. 15A and 15B illustrate that the diverter 100 shown in FIGS. 14A and 14B can have an opening 100o when the diverter 100 is attached to the endoscope 300. FIGS. 15A and 15B illustrate that the opening 100o can be a section of the opening to the cavity 104. The opening 100o can be, for example, be an opening between the base of the diverter 100 and the handle of the endoscope 300. As another example, the opening 100o can be hole in the body 101 of the diverter 100. FIG. 15A illustrates that the diverter 100 can split the flow 502 of gas and/or particles from the opening 314o into a first flow 502a and a second flow 502b. The first flow 502a can flow out of the cavity 104 into the suction channel 390, for example, though through the suction button 316. The first flow 502a of gas and/or particles can, for example, then be suctioned to the suction source and/or to a filter. The second flow 502b of gas and/or particles can flow out of the cavity 104 between a base of the diverter 100 and the endoscope 300 (e.g., through the opening 100o) into the space (e.g., room) that the endoscope 300 is in. The gas and/or particles in the second flow 502b can be aerosolized but the second flow 502b is shown to be directed away from the user 500.

FIGS. 15A and 15B illustrate that the diverter 100 can prevent the aerosolization of gas and/or particles from the first flow 502a outside of the cavity 104. FIGS. 15A and 15B illustrate that the diverter 100 can allow the aerosolization of gas and/or particles from the second flow 502b outside of the cavity 104 but can direct such aerosolization away from the user 500.

FIGS. 15A and 15B illustrate that when the diverter 100 is attached to an endoscope (e.g., the endoscope 300), the diverter 100 can divert the flow gas and/or particles ejected from the opening 314o in a first direction (e.g., the direction of the first flow 502a) and in a second direction (e.g., the direction of the second flow 502b). FIGS. 15A and 15B illustrate that the diverter 100 can move the point at which the gas and/or particles ejected from the opening 314o are released into the space (e.g., the room) that the endoscope 300 is in, for example, from the opening 314o to the opening 100o.

FIGS. 14A-15B illustrate that there can be sufficient suction through the suction button 316 into the cavity 104 to take up the gas and/or particles that are released from the opening 314.

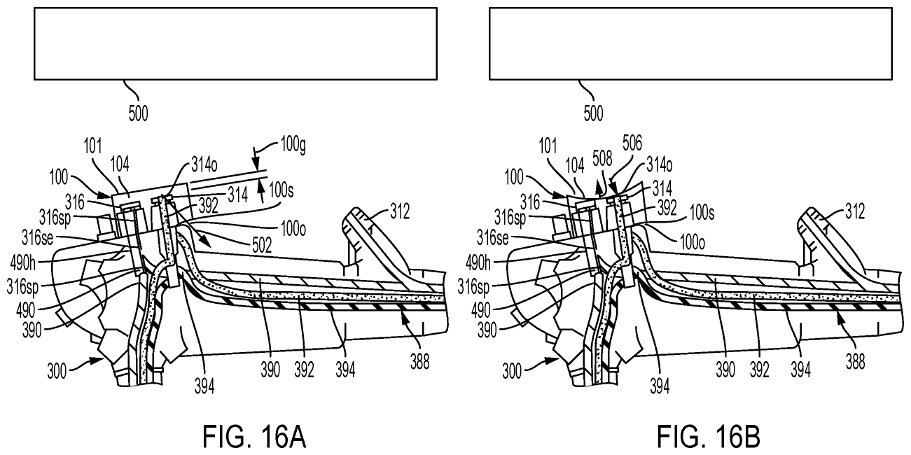

FIGS. 16A and 16B illustrate that the diverter 100 shown in FIGS. 15A and 15B can be used with an endoscope 300 that does not have any suction in the cavity 104 (e.g., that does not leak suction through the suction button 316). In such cases, FIGS. 16A and 16B illustrate that the diverter 100 can divert the flow 502 of gas and/or particles from the opening 314o to the opening 100o so that the flow 502 of gas and/or particles can flow away from the user 500.

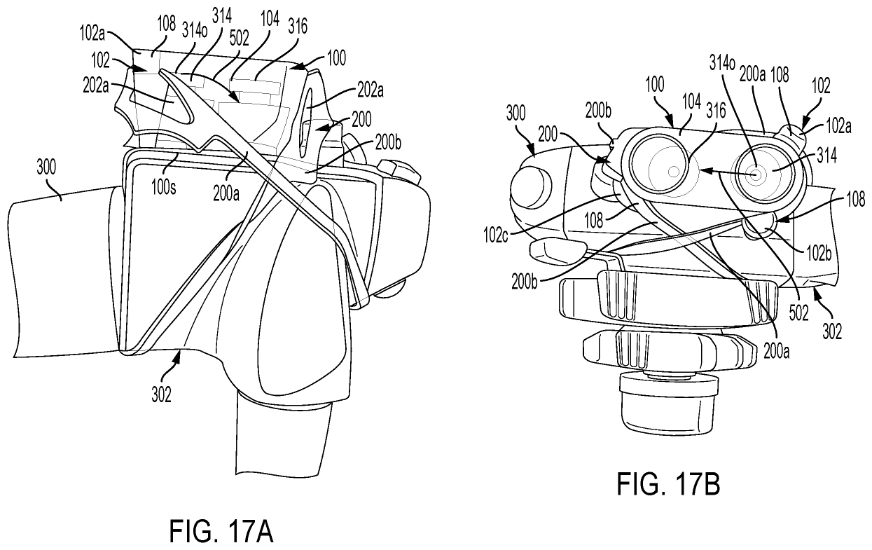

FIGS. 17A and 17B illustrate that the diverter 100 of FIGS. 1-3 can be removably attached to the handle of the endoscope 300 with a first strap 200a and a second strap 200b. FIG. 17A illustrates that the first strap 200a can secure a first longitudinal end of the diverter 100 to the endoscope 300 and that the second strap 200 can secure a second longitudinal end of the diverter 100 to the endoscope 300, for example, such that the seal 100s is formed between the base of the diverter 100 and the endoscope 300. FIGS. 17A and 17B illustrate that the first and second straps 200a, 200b can pull in opposite directions which can increase the strength of the seal 100s. FIG. 17A illustrate that the first strap 200a can cross over the second strap 200b or vice versa. FIGS. 17A and 17B illustrate that the first and second straps 200a, 200b can each extend around the handle of the endoscope 300. FIGS. 17A and 17B illustrate that the first connector 102a can extend through the first opening 202a of the first strap 200a. FIGS. 17A and 17B illustrate that the first strap 200a can be in the groove 108 of the first connector 102a and in the groove 108 of the second connector 102b, and that the second strap 200b can be in the in the groove 108 of the third connector 102c. FIGS. 17A and 17B illustrate that the diverter 100 can be transparent. Without the first strap 200a and/or the second strap 200b, the diverter 100 can have the opening 100o. The first strap 200a and/or the second strap 200b can thereby be used to selectively open and/or selectively close the opening 100o. When the diverter 100 is removably secured to the endoscope 300 with the first strap 200a and/or with the second strap 200b, FIGS. 17A and 17B illustrate that the opening 100o can be closed. When the diverter 100 is removably secured to the endoscope 300 without the first strap 200a and/or without the second strap 200b, the opening 100o can be open, for example, as shown in FIGS. 15A and 15B.

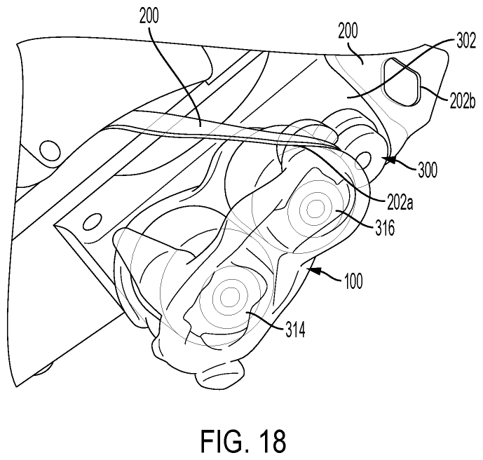

FIG. 18 illustrates that the diverter 100 can be removably secured to the handle with one strap 200.

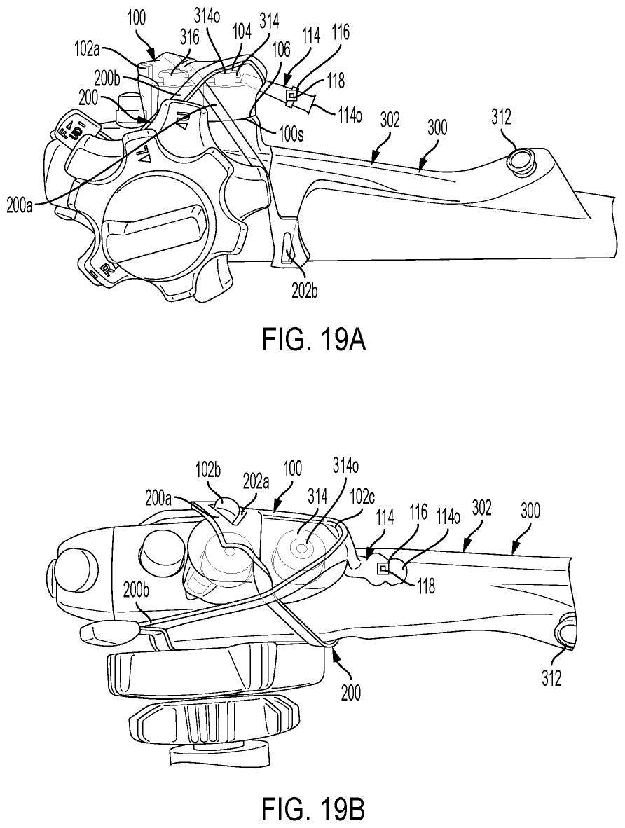

FIGS. 19A and 19B illustrate that the diverter 100 can have a connector 114. The connector 114 can be fixedly attached or removably attached to the diverter 100. The connector 114 can be attached to or integrated with the diverter 100. For example, FIGS. 19A and 19B illustrate that the connector 114 can be integrated with the diverter 100. The connector 114 can be, for example, integrally formed with the diverter 100 during a molding or 3D printing process.

FIGS. 19A and 19B illustrate that the connector 114 can be, for example, a luer connector. A tube can attach to the connector 114. For example, a first end of the tube can attach to the connector 114 and second end of the tube can be free (e.g., not attached to anything), can be attached to the working channel valve 312 (e.g., to the connect the cavity 104 to the suction channel 390 via the working channel valve 312), or can be attached to a filter. The connector 114 can, for example, divert the flow 502 of gas and/or particles out of the cavity and/or away from the user 500.

FIGS. 19A and 19B illustrate that the connector 114 can have a valve 116 and a control 118 connected to the valve 116. The valve 116 can be, for example, a stopcock that the control 118 can open and close, for example to control the flow of gas and/or particles out of the cavity 104 through the connector 114. The control 118 can be, for example, a rotatable knob or wheel. When the connector 114 is not connected to a tube and/or to a filter, the valve 116 can be closed, for example, so that the flow 502 of gas and/or particles from the opening 314o can be prevented from flowing out of the cavity 104 through the connector 114 through the opening 114o at the end of the connector 114. As another example, when the connector 114 is not connected to a tube and/or to a filter, the valve 116 can be open, for example, so that the flow 502 of gas and/or particles from the opening 314o can be allowed to flow out of the cavity 104 through the connector 114, for example, to direct the flow 502 of gas and/or particles away from the user 500.

FIGS. 19A and 19B illustrate that when the diverter 100 is removably attached to the endoscope 300, the straps 200 can extend over the top of the diverter 100 without occluding the opening 314o. FIGS. 19A and 19B illustrate that the first strap 200a can extend under the second strap 200b, that the first strap 200a can be connected to the second connector 102b, and that the second strap 200b can be connected to the third connector 102c. FIGS. 19A and 19B illustrate that not all of the connects 102 may be used to releasably secure the diverter 100 to the endoscope 300. As another example, all of the connectors 102 can be used to releasably secure the diverter 100 to the endoscope 300.

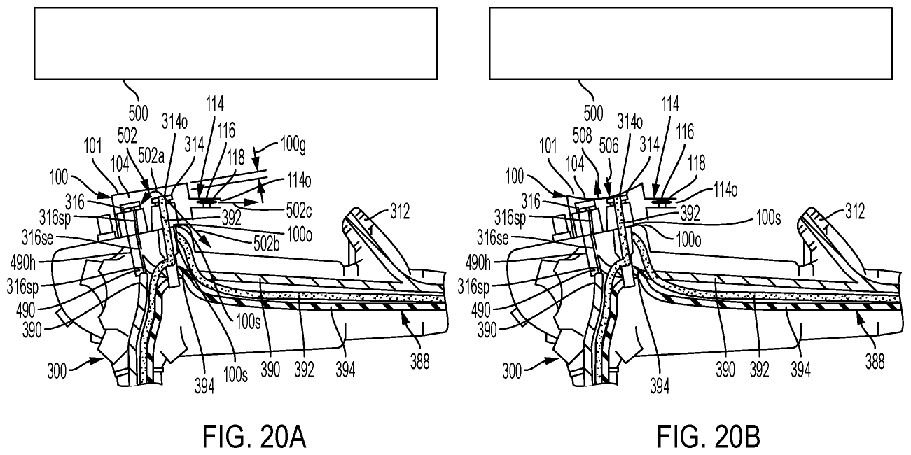

FIG. 20A illustrates that the diverter 100 can split the flow 502 of gas and/or particles from the opening 314o into a first flow 502a, a second flow 502b, and a third flow 502c, or any combination thereof. The first flow 502a can flow out of the cavity 104 into the suction channel 390, for example, though through the suction button 316. The first flow 502a of gas and/or particles can, for example, then be suctioned to the suction source and/or to a filter. The second flow 502b of gas and/or particles can flow out of the cavity 104 between a base of the diverter 100 and the endoscope 300 (e.g., through the opening 100o) into the space (e.g., room) that the endoscope 300 is in. The gas and/or particles in the second flow 502b can be aerosolized but the second flow 502b is shown to be directed away from the user 500. The third flow 502c of gas and/or particles can flow out of the cavity 104 through the connector 114 (e.g., through the valve 116 and the opening 114o) into the space (e.g., room) that the endoscope 300 is in.

FIG. 20A illustrates that the diverter 100 can divert flow through the suction button 316 (e.g., through the space 316sp) via the first flow 502a, through the base of the diverter 100 (e.g., through the opening 100o and/or through the seal 100s) via the second flow 502b, and through the connector 114 (e.g., through the opening 114o) via the third flow 502c, or any combination thereof. FIG. 20A illustrates, for example, that the diverter 100 can split the opening 314o into the opening of the space 316sp, to the opening 100s, and to the opening 114o, or any combination thereof.

FIG. 20A illustrates that the first flow 502, the second flow 502b, and the third flow 502c can each be selectively increased or decreased. FIG. 20A illustrates the straps 200 of FIGS. 19A and 19B transparent so that the features shown in FIG. 20A can be more easily seen. For example, if the connector 114 is closed (e.g., by closing the valve 116 via the control 118), flow can be increased to the suction button 316 and to the base of the diverter 100 (e.g., to the opening 100o and/or through the seal 100s), and if the base of the diverter 100 is sealed tighter (e.g., if the seal 100s is tighter), for example, by using tighter bands and the connector 114 is closed, flow can be increased to the suction button 316.

The first flow 502a can be increased, for example, by terminating or decreasing the second flow 502b and/or the third flow 502c. The first flow 502a can be decreased, for example, by activating or increasing the second flow 502b and/or the third flow 502c.

The second flow 502b can be increased, for example, by loosening the first strap 200a and/or by loosening the second strap 200b. The straps 200 can be loosened, for example, by wrapping the straps 200 around the endoscope 300 less tightly or by using larger straps 200. The second flow 502b can be decreased, for example, by tightening the first strap 200a and/or by tightening the second strap 200b. The straps 200 can be tightened, for example, by wrapping the straps 200 around the endoscope 300 more tightly or by using smaller straps 200.

The third flow 502c can be increased, for example, by opening the valve 116 via the control 118. The third flow 502 can be decreased, for example, by closing the valve 116 via the control 118.

FIG. 20A illustrates that each flow (e.g., the first flow 502a, the second flow 502b, and the third flow 502c) can be selectively activated and deactivated.

The first flow 502a may be deactivated, for example, by pressing the suction button 316 and/or by placing the diverter 100 on an endoscope 300 that does not leak suction though the suction button 316. As additional examples, if the connector 114 is closed (e.g., by closing the valve 116 via the control 118), flow can be diverted to the suction button 316 and base of the diverter 100 (e.g., to the opening 100o and/or through the seal 100s), and if the base is sealed tighter, for example, by using tighter bands and the connector 114 is closed, flow can be diverted to the suction button 316.

FIGS. 20A and 20B illustrate that the user can use the gas/water button 314 and the suction button 316 while the diverter 100 is attached to the endoscope 300.

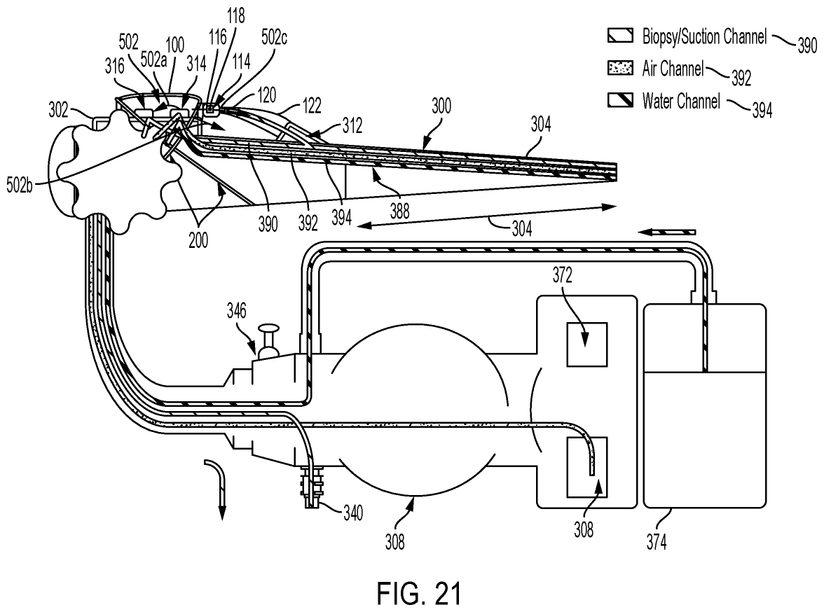

FIG. 21 illustrates that a filter 120 can be attached to or integrated with the connector 114. FIG. 21 illustrates that a tube 122 can connect the connector 114 to the working channel valve 312. For example, FIG. 21 illustrates that a first end of the tube 122 can connect to the connector 114, and that a second end of the tube 122 can connect to the working channel valve 312. Such an arrangement can direct the flow of gas and/or particles from the cavity 104 into the suction channel 390 through the connector 114, through the filter 120, and through the tube 120.

The diverter 100 can across different endoscope designs. The diverter 100 can be compatible with different endoscopes, for example, with OLYMPUS endoscopes and with FUJIFILM endoscopes. As another example, to make the diverter 100 compatible across different endoscopes, straps 200 can be used. The straps 200 can, for example, enable the diverter 100 to be compatible with a variety of different endoscopes and with a variety of different button configurations. The straps 200 can be used to modify or adjust the fit between the base of the diverter 100 and an endoscope (e.g., the endoscope 300) and to prevent the diverter 100 from falling off as the endoscope (e.g., the endoscope 300) is torqued and moved around. The straps 200 can, for example, keep the diverter 100 in place.

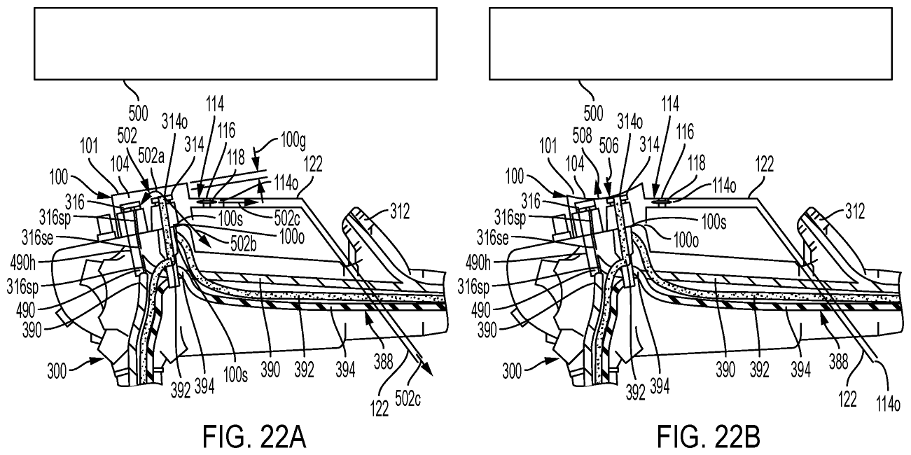

FIGS. 22A and 22B illustrate that a tube (e.g., the tube 122) can be attached to the connector 114 to divert the flow of the third flow 502c of gas and/or particles away from the user 500, away from the user's team members, and/or away from the patient. FIGS. 22A and 22B illustrate that the first end of the tube 122 can be connected to the connector 114 and that the second end of the tube 122 can be free (e.g., not connected to anything). FIGS. 22A and 22B illustrate that the tube 122 can extend over the endoscope 300. The tube 122 can be, for example, 0.05 m to 10 m long, including every 0.01 m increment within this range (e.g., 0.05 meters, 0.10 meters, 1 meter, 10 meters). FIGS. 22A and 22B illustrate that the third flow 502c of gas and/or particles can extend through the tube 122. As another example, the second of the tube 122 may not be free. In such cases, the second end of the tube 122 can be, for example, connected to a filter that can, for example, clean the third flow 502c of gas of infectious particles.

The diverter 100 can be disposable or reusable.

The sealing strap 200 can be disposable or reusable.

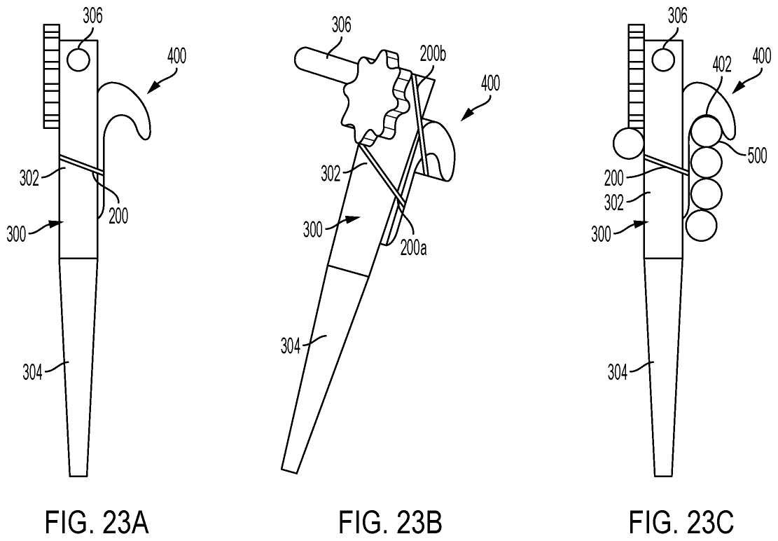

FIGS. 23A and 23B illustrate a variation of an ergonomic rest 400 (also referred to as an ergonomic enabling device and as a rest). The ergonomic rest 400 can be tethered to an endoscope (e.g., the endoscope 300) using one or more straps 200. The ergonomic rest 400 can be designed to facilitate recruitment of muscle groups in the user's arm (e.g., upper arm) to reduce weight bearing. For example, FIG. 23A illustrates that a strap 200 (e.g., one strap 200) can removably connect the rest 400 to the endoscope 300 (e.g., to the handle of the endoscope 300). As another example, FIG. 23B illustrates that two straps (e.g., a first strap 200a and a second strap 200b) can removably connect the rest 400 to the endoscope 300 (e.g., to the handle of the endoscope 300).

FIG. 23C illustrates a user 500 operating the endoscope 300 with a rest 400. For example, with the five solid white circles representing the user's fingers, where the thumb is on the left side of FIG. 23C and where the index finger is in a groove 402 of the rest 400 and the other three fingers are below the index finger in FIG. 23C.

The ergonomic rest 400 can comprise a soft material such as silicone and also other materials can be used. The material selection in this case can aid to reduce the pressure and tension felt in the user's hand.

The ergonomic rest 400 can be positioned in a number of different locations and affixed to the endoscope (e.g., the endoscope 300) by use of tethers (e.g., the straps 200 as shown in FIGS. 4 and 5).

The ergonomic rest 400 can have surface features added to the material to aid with gripping.

The ergonomic rest 400 can be disposable or reusable.

The features described and shown herein can be combined in any combination. For example, FIGS. 1-22B illustrate that a flow diverter 100 can be removably securable to a medical device (e.g., the endoscope 300). The flow diverter can have a body 101 and a cavity 104. When the body is removably secured to the medical device and when a flow of gas (e.g., the flow 502 of gas and/or particles) flows from the medical device into the cavity, the flow of gas can be divertible from a first direction to a second direction. When the body is removably secured to the medical device and when the flow of gas flows from the medical device into the cavity, the flow of gas can be divertible out of the cavity. The features described and shown herein can be combined in any combination. For example, FIGS. 1-22B illustrate that a flow diverter 100 can be removably securable to a medical device (e.g., the endoscope 300). The flow diverter can have a body and a cavity. The body and the cavity have a non-deflected configuration and a deflected configuration. The body can have a non-deflected configuration and the body can have a deflected configuration. The cavity can have a non-deflected configuration and a deflected configuration. Exemplary non-deflected configurations of the body and the cavity are shown in FIGS. 1-3, 6, 14A, 15A, 16A, 17A-20A, 21, and 22A. Exemplary deflected configurations of the body and the cavity are shown, for example, in FIGS. 14B, 15B, 16B, 20B, and 22B. These figures show that the volume of the cavity 104 can be smaller when the body and the tube are in the deflected configuration than when the body and the tube are in the non-deflected configuration. When the body is removably secured to the medical device and when the body and the cavity are in the non-deflected configuration, a first flow path (e.g., the path of the first flow 502a, the flow path that the first flow 502a flows through) can extend from the cavity, a second flow path (e.g., the path of the second flow 502b, the flow path that the second flow 502b flows through) can extend from the cavity, and/or a third flow path (e.g., the path of the third flow 502c, the flow path that the third flow 502c flows through) can extend from the cavity. The first direction can be away from the medical device, and the second direction can be toward the medical device. The first direction can be opposite the second direction. The first direction can be away from the medical device, and the second direction can be away from the medical device. The second direction can be at an angle of 70 degrees to 180 degrees relative to the first direction. When the body is removably secured to the medical device and when the flow of gas flows from the medical device into the cavity, the flow of gas can be divertible out of the cavity toward the medical device. When the body is removably secured to the medical device and when the flow of gas flows from the medical device into the cavity, the flow of gas can be divertible out of the cavity into the medical device. When the body is removably secured to the medical device and when the flow of gas flows from the medical device into the cavity, the flow of gas can be divertible out of the cavity into a suction channel in the medical device. When the body is removably secured to the medical device and when the flow of gas flows from the medical device into the cavity, the flow of gas can be divertible out of the cavity into a suction channel in the medical device through a channel (e.g., the space 316sp) that can extend through a suction button. When the body is removably secured to the medical device and when the flow of gas flows from the medical device into the cavity, the flow of gas can be divertible out of the cavity through an opening (e.g., the opening 100o) between a base of the body and a surface of the endoscope. The diverter can have a connector (e.g., the connector 114). When the body is removably secured to the medical device and when the flow of gas flows from the medical device into the cavity, the flow of gas can be divertible out of the cavity through the connector. When the body is removably secured to the medical device and when the flow of gas flows from the medical device into the cavity, the flow of gas can be divertible out of the cavity away from the medical device. When the body is removably secured to the medical device and when the flow of gas flows from the medical device into the cavity, the flow of gas from the medical device can be divertible into the medical device through a button or through a channel that can extend through a button. When the body is removably secured to the medical device, a first button (e.g., the gas/water button 314) and a second button (e.g., the suction button 316) can be in the cavity. When the body is removably secured to the medical device and when the flow of gas flows from the medical device into the cavity, the flow of gas from the medical device can be divertible from the first button to the second button. When the body is removably secured to the medical device and when the flow of gas flows from the medical device into the cavity, the flow of gas from the medical device can be ejected through an opening (e.g., the opening 314o) in the first button and can be suctionable into a suction channel in the medical device through the second button or through a channel that can extend through or along the second button. The first button can be a gas/water button (e.g., the gas/water button 314), and the second button can be a suction button (e.g., the suction button 316). When the body is detached from the medical device and when the flow of gas flows from the medical device, the flow of gas can flow in the first direction. When the body is removably secured to the medical device and when the flow of gas flows from the medical device into the cavity, the flow of gas can be divertible from a first direction to a second direction via the body. When the body is removably secured to the medical device and when the flow of gas flows from the medical device into the cavity, the flow of gas can be divertible out of the cavity via the body. The body can be removably securable to the medical device with a strap (e.g., a strap 200). The body can be removably securable to the medical device with a first strap (e.g., the first strap 200a) and a second strap (e.g., the second strap 200b). The medical device can be an endoscope (e.g., the endoscope 300).

The features described and shown herein can be combined in any combination. For example, FIGS. 1-22B illustrate that a flow diverter 100 can be removably securable to a medical device (e.g., the endoscope 300). The flow diverter can have a body 101 and a cavity 104. When the body is removably secured to the medical device and when a flow of gas (e.g., the flow 502 of gas and/or particles) flows from the medical device into the cavity, the flow of gas can be selectively divertible into a first flow path (e.g., the path of the first flow 502a, the flow path that the first flow 502a flows through), into a second flow path (e.g., the path of the second flow 502b, the flow path that the second flow 502b flows through), and/or into a third flow path (e.g., the path of the third flow 502c, the flow path that the third flow 502c flows through). When the body is removably secured to the medical device, a first button (e.g., the gas/water button 314) and a second button (e.g., the suction button 316) can be in the cavity. The first button can be a gas/water button (e.g., the gas/water button 314), and the second button can be a suction button (e.g., the suction button 316). When the flow of gas flows from the medical device into the cavity, the flow of gas can flow into the cavity from the first button. The first flow path can extend through the second button or through a channel (e.g., the space 316sp) that can extend through or along the second button. The second flow path can extend through an opening (e.g., the opening 100o) between a base of the body and a surface of the medical device. The flow diverter can have a connector (e.g., the connector 114). The third flow path can extend through the connector. A flow along the first flow path can be increasable by decreasing or terminating a flow along the second flow path, and/or the flow along the first flow path can be increasable by decreasing or terminating a flow along the third flow path. A flow along the first flow path can be increasable by partially or fully closing the second flow path, and/or the flow along the first flow path can be increasable by partially or fully closing the third flow path. A flow along the second flow path can be increasable by decreasing or terminating a flow along the third flow path. A flow along the second flow path can be increasable by partially or fully closing the third flow path. A flow along the third flow path can be increasable by decreasing or terminating a flow along the second flow path. A flow along the third flow path can be increasable by partially or fully closing the second flow path. A flow along the first flow path can be decreasable by increasing a flow along the second flow path, and/or the flow along the first flow path can be decreasable by increasing a flow along the third flow path. A flow along the first flow path can be decreasable by partially or fully opening the second flow path, and/or the flow along the first flow path can be decreasable by partially or fully opening the third flow path. A flow along the second flow path can be decreasable by increasing a flow along the third flow path. A flow along the second flow path can be decreasable by partially or fully opening the third flow path. A flow along the third flow path can be decreasable by increasing a flow along the second flow path. A flow along the third flow path can be decreasable by partially or fully opening the second flow path. The body can be removably securable to the medical device with a strap (e.g., the strap 200). The body can be removably securable to the medical device with a first strap (e.g., the first strap 200a) and a second strap (e.g., the second strap 200b). The medical device can be an endoscope 300.

The features described and shown herein can be combined in any combination. For example, FIGS. 1-22B illustrate that a flow diverter 100 can be removably securable to a medical device (e.g., the endoscope 300). The flow diverter can have a body 101 and a cavity 104. When the body is removably secured to the medical device and when a flow of gas flows from the medical device into the cavity, the flow of gas can be selectively divertible into a first flow path (e.g., the path of the first flow 502a, the flow path that the first flow 502a flows through), into a second flow path (e.g., the path of the second flow 502b, the flow path that the second flow 502b flows through), and/or into a third flow path (e.g., the path of the third flow 502c, the flow path that the third flow 502c flows through).

The features described and shown herein can be combined in any combination. For example, FIGS. 1-22B illustrate that a flow diverter 100 can be removably securable to a medical device (e.g., the endoscope 300). The flow diverter can have a body and a cavity. When the body is removably secured to the medical device, a first flow path (e.g., the path of the first flow 502a, the flow path that the first flow 502a flows through) can be openable and closable, a second flow path (e.g., the path of the second flow 502b, the flow path that the second flow 502b flows through) can be openable and closable, and/or a third flow path (e.g., the path of the third flow 502c, the flow path that the third flow 502c flows through) can be openable and closable. When the body is removably secured to the medical device, a first button (e.g., the gas/water button 314) and a second button (e.g., the suction button 316) can be in the cavity. The first button can be a gas/water button (e.g., the gas/water button 314), and the second button can be a suction button (e.g., the suction button 316). The first flow path can be closable by pressing the second button, and the first flow path can be openable by depressing (e.g., releasing) the second button. The second flow path can be closable by increasing a force exerted against the body by a strap (e.g., the strap 200), and the second flow path can be openable by decreasing the force exerted against the body by the strap. The flow diverter can have a connector (e.g., the connector 114). The third flow path can be closable via the connector (e.g., by closing the connector 114), and the third flow path can be openable via the connector (e.g., by opening the connector 114).

The features described and shown herein can be combined in any combination. For example, FIGS. 1-22B illustrate that a flow diverter 100 can be removably securable to a medical device (e.g., the endoscope 300). The flow diverter can have a body and a cavity. When the body is removably secured to the medical device, the body can be configured to divert a flow of gas (e.g., the flow 502 of gas and/or particles) from the medical device from a first direction into a second direction, and/or when the body is removably secured to the medical device, the body can be configured to divert the flow of gas the flow of gas out of the cavity.

The features described and shown herein can be combined in any combination. For example, FIGS. 1-22B illustrate that a flow diverter 100 can be removably securable to a medical device (e.g., the endoscope 300). The flow diverter can have a body and a cavity. When the body is removably secured to the medical device, the body can be configured to divert (e.g., split) a flow of gas (e.g., the flow 502 of gas and/or particles) from the medical device into a first flow (e.g., the first flow 502a), into a second flow (e.g., the second flow 502b), and/or into a third flow (e.g., the third flow 502c). The flow diverter can be configured to prevent aerosolization of gas and/or particles from the medical device. The flow diverter can be removably attached to the medical device via a strap (e.g., the strap 200).

The features described and shown herein can be combined in any combination. For example, FIGS. 1-22B illustrate a method of diverting a flow of gas (e.g., the flow 502 of gas and/or particles) from a medical device (e.g., the endoscope 300). The method can include attaching a flow diverter 100 to the medical device.

The features described and shown herein can be combined in any combination. For example, FIGS. 1-22B illustrate a method of inhibiting or preventing aerosolization comprising attaching a flow diverter 100 to a medical device (e.g., the endoscope 300).

The features described and shown herein can be combined in any combination. For example, FIGS. 23A-23C illustrate an ergonomic rest 400. The ergonomic rest can be attachable to an endoscope (e.g., the endoscope 300), and the ergonomic rest can be configured to prevent overuse injuries.

Any of the features disclosed, contemplated, and/or illustrated herein can be combined with any commercially available medical equipment (e.g., with any commercially available endoscopic equipment).

The claims are not limited to the exemplary variations shown in the drawings, but instead may claim any feature disclosed or contemplated in the disclosure as a whole. Any elements described herein as singular can be pluralized (i.e., anything described as “one” can be more than one). Any species element of a genus element can have the characteristics or elements of any other species element of that genus. Some elements may be absent from individual figures for reasons of illustrative clarity. The above-described configurations, elements or complete assemblies and methods and their elements for carrying out the disclosure, and variations of aspects of the disclosure can be combined and modified with each other in any combination, and each combination is hereby explicitly disclosed. All devices, apparatuses, systems, and methods described herein can be used for medical (e.g., diagnostic, therapeutic or rehabilitative) or non-medical purposes. The words “may” and “can” are interchangeable (e.g., “may” can be replaced with “can” and “can” can be replaced with “may”). Any range disclosed can include any subrange of the range disclosed, for example, a range of 1-10 units can include 2-10 units, 8-10 units, or any other subrange. Any phrase involving an “A and/or B” construction can mean (1) A alone, (2) B alone, (3) A and B together, or any combination of (1), (2), and (3), for example, (1) and (2), (1) and (3), (2) and (3), and (1), (2), and (3). The term about can include any tolerance that would be understood by one or ordinary skill in the art, for example, plus or minus 5% of the stated value.

We claim:

1. A flow diverter removably securable to a medical device, the flow diverter comprising:
a body and a cavity,
wherein when the body is removably secured to the medical device and when a flow of gas flows from the medical device into the cavity, the flow of gas is divertible from a first direction to a second direction, and/or
wherein when the body is removably secured to the medical device and when the flow of gas flows from the medical device into the cavity, the flow of gas is divertible out of the cavity.

2. The flow diverter of claim 1, wherein when the body is removably secured to the medical device and when the flow of gas flows from the medical device into the cavity, the flow of gas is divertible out of the cavity into a suction channel in the medical device.

3. The flow diverter of claim 1, wherein when the body is removably secured to the medical device and when the flow of gas flows from the medical device into the cavity, the flow of gas is divertible out of the cavity through an opening between a base of the body and a surface of the medical device.

4. The flow diverter of claim 1, further comprising a connector, wherein when the body is removably secured to the medical device and when the flow of gas flows from the medical device into the cavity, the flow of gas is divertible out of the cavity through the connector.

5. The flow diverter of claim 1, wherein when the body is removably secured to the medical device and when the flow of gas flows from the medical device into the cavity, the flow of gas from the medical device is divertible into the medical device through a button or through a channel that extends through a button.

6. The flow diverter of claim 1, wherein when the body is removably secured to the medical device, a first button and a second button are in the cavity, and wherein when the body is removably secured to the medical device and when the flow of gas flows from the medical device into the cavity, the flow of gas from the medical device is ejected through an opening in the first button and is suctionable into a suction channel in the medical device through the second button or through a channel that extends through or along the second button.

7. The flow diverter of claim 6, wherein the first button comprises a gas/water button, wherein the second button comprises a suction button, wherein the body is removably securable to the medical device with a strap, and wherein the medical device comprises an endoscope.

8. A flow diverter removably securable to a medical device, the flow diverter comprising:
a body and a cavity,
wherein when the body is removably secured to the medical device and when a flow of gas flows from the medical device into the cavity, the flow of gas is selectively divertible into a first flow path, into a second flow path, and/or into a third flow path.

9. The flow diverter of claim 8, wherein when the body is removably secured to the medical device, a first button and a second button are in the cavity, and wherein when the flow of gas flows from the medical device into the cavity, the flow of gas flows into the cavity from the first button.

10. The flow diverter of claim 9, wherein the first flow path extends through the second button or through a channel that extends through or along the second button.

11. The flow diverter of claim 10, wherein the second flow path extends through an opening between a base of the body and a surface of the medical device.

12. The flow diverter of claim 11, further comprising a connector, wherein the third flow path extends through the connector.

13. The flow diverter of claim 12, wherein a flow along the first flow path is increasable by decreasing or terminating a flow along the second flow path, wherein the flow along the first flow path is increasable by decreasing or terminating a flow along the third flow path, wherein a flow along the second flow path is increasable by decreasing or terminating a flow along the third flow path, and wherein a flow along the third flow path is increasable by decreasing or terminating a flow along the second flow path.

14. The flow diverter of claim 13, wherein the body is removably securable to the medical device with a strap, and wherein the medical device comprises an endoscope.

15. The flow diverter of claim 9, wherein the first flow path is closable by pressing the second button, and wherein the first flow path is openable by depressing the second button.

16. The flow diverter of claim 15, wherein the second flow path is closable by increasing a force exerted against the body by a strap, and wherein the second flow path is openable by decreasing the force exerted against the body by the strap.

17. The flow diverter of claim 16, further comprising a connector, wherein the third flow path is closable by via the connector, and wherein the third flow path is openable via the connector.

18. A method of inhibiting or preventing aerosolization via a flow diverter attached to a medical device, wherein the flow diverter comprises a body, a cavity, and a connector, and wherein the medical device comprises a first button and a second button, the method comprising:
removably positioning the flow diverter over the first button and the second button; and
diverting a gas that flows into the cavity into a first flow path, into a second flow path, and/or into a third flow path, wherein the first flow path extends through the second button or through a channel that extends through or along the second button, wherein the second flow path extends through an opening between a base of the body and a surface of the medical device, and wherein the third flow path extends through the connector.

19. The method of claim 18, further comprising:
closing the first flow path by pressing the second button;
opening the first flow path by depressing the second button;
closing the second flow path by increasing a force exerted against the body by a strap;
opening the second flow path by decreasing the force exerted against the body by the strap;
closing the third flow path via the connector; and
opening the third flow path via the connector.

20. The method of claim 18, wherein the medical device comprises an endoscope, and wherein the method further comprises:
increasing a flow along the first flow path by decreasing or terminating a flow along the second flow path;
increasing the flow along the first flow path by decreasing or terminating the flow along the second flow path;
increasing the flow along the first flow path by decreasing or terminating a flow along the third flow path;
increasing the flow along the second flow path by decreasing or terminating the flow along the third flow path; and
increasing the flow along the third flow path by decreasing or terminating the flow along the second flow path.
